(19) United States
(12) Patent Application Publication
(10) Pub. No.: US 2011/0097287 A1
(43) Pub. Date: Apr. 28, 2011

(54) COSMETIC COMPOSITION COMPRISING A CATIONIC SURFACTANT COMPOUND, NOVEL COMPOUNDS, USE AS CONDITIONER AND COSMETIC TREATMENT METHOD

(75) Inventors: Laure Ramos-Stanbury, Sceaux (FR); Maria Dalko, Gif S/yvette (FR); Gilles Genain, Paris (FR); Estelle Mathonneau, Paris (FR); Virginie Le Chaux, Franconville (FR)

(73) Assignee: L'OREAL, Paris (FR)

(21) Appl. No.: 12/865,880

(22) PCT Filed: Feb. 3, 2009
(86) PCT No.: PCT/FR2009/050164

§ 371 Date: Dec. 22, 2010

Related U.S. Application Data
(60) Provisional application No. 61/027,836, filed on Feb. 12, 2008.

(30) Foreign Application Priority Data
Feb. 4, 2008 (FR) 0850662

(51) Int. Cl. A61K 8/40; C07C 69/003; C07C 69/22; A61Q 5/00; A61Q 17/04

(52) U.S. Cl. 424/60; 424/70.28; 560/129; 560/205; 560/67; 424/70.9; 424/70.22; 424/70.21; 424/59

(57) ABSTRACT

The present patent application relates to cosmetic compositions comprising cationic compounds of quaternary ammonium esters derived from 2-[2-(diethylamino)ethoxy]ethanol type, to the use of these cationic compounds, in particular as conditioning agent, to a cosmetic treatment method, in particular a hair treatment method, employing them and to the novel cationic compounds.

The present invention relates to novel surface-active cationic compounds of quaternary ammonium esters of 2-[2-(diethylamino)ethoxy]ethanol derivatives type, to the cosmetic compositions comprising them and to their use, in particular for cosmetically treating the hair.

It is well known that hair which has been sensitized, in particular damaged and/or embrittled, to various degrees under the action of atmospheric agents or under the action of mechanical and/or chemical treatments, such as dyeing operations, bleaching operations or perming operations, is often difficult to disentangle and to style and also lacks softness.

Cosmetic compositions comprising conditioning cationic surfactants, such as those described in US 2006/0078529, have already been proposed for the treatment of keratinous substances and in particular the hair. However, such compositions still do not have the desired cosmetic qualities, in particular in terms of sensorial properties, in particular of feel of the hair after treatment.

In point of fact, the Applicant Company has now discovered, unexpectedly and surprisingly, that some very specific cationic compounds can contribute advantageous conditioning properties to the hair, in particular relating to the improvement in the suppleness of the hair when the composition is applied to wet hair and then the achievement, after rinsing, of hair still exhibiting an improved suppleness, the hair thus being less stiff. This rendering of the hair supple is particularly noteworthy with the compositions according to the invention.

Furthermore, in addition to the suppleness, these compositions also make it possible to improve the disentangling, the smoothing, the ability to be combed and the manageability of the hair; the shaping of the hair is easier and the feel of the hair is very pleasant and fluid.

It has also been found that, in the presence of fatty alcohols, the cationic surfactants according to the invention do not destabilize the composition, in particular the emulsion, comprising them, contrary to what might be observed with some nonquaternary aminated cationic surfactants of the prior art, in particular cationic surfactants comprising tertiary amines. Thus, a particularly surprising advantage of the present invention lies in the possibility of formulating fatty alcohols, in particular C₁₆-C₁₈ fatty alcohols, in the presence of aminated cationic surfactants.

In addition, the compounds according to the invention can be easily conveyed in aqueous cosmetic media, which facilitates the use thereof.

Thus, the subject-matter of the present invention is a method for the cosmetic treatment of keratinous substances comprising the application, to the said substances, of a cosmetic composition comprising, in a physiologically acceptable medium, at least one compound of formula (I) as defined below.

Another subject-matter of the invention is the use of at least one compound of formula (I) as defined below as conditioning agent, in particular in a hair cosmetic composition.

Another subject-matter of the invention is a cosmetic composition comprising at least one compound of formula (I) as defined below and a cosmetically acceptable medium comprising at least one standard cosmetic ingredient chosen in particular from propellants, water, carbon-comprising oils, C₈-C₄₀ esters, C₈-C₄₀ acids, C₁-C₄₀ alcohols, organic solvents, nonionic surfactants, cationic surfactants, anionic surfactants, amphoteric surfactants, zwitterionic surfactants, sunscreens, moisturizing agents, antidandruff agents, antioxidants, reducing agents, oxidation bases, couplers, oxidizing agents, direct dyes, hair-straightening agents, pearlescent and opacifying agents, plasticizing or coalescence agents, hydroxy acids, pigments, fillers, silicones, polymeric or nonpolymeric thickeners, emulsifiers or polymers, in particular conditioning or styling polymers.

Another subject-matter of the invention is a compound of formula (Ia) as defined below and the cosmetic compositions comprising it.

The compounds according to the invention thus correspond to the formula (I):

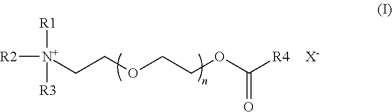

in which:

n is an integer between 1 and 10;

R1, R2 and R3 denote, independently of one another, a saturated or unsaturated and linear, cyclic or branched carbon-comprising group, in particular a hydrocarbon group, comprising from 1 to 22 carbon atoms (in particular alkyl or alkenyl) and optionally substituted by one or more hydroxyl (—OH) and/or amino (—NRR′) radicals, with R and R′ chosen, independently of one another, from H and C₁-C₆ alkyl; or else

R1 denotes a saturated or unsaturated and linear, cyclic or branched carbon-comprising group, in particular a hydrocarbon group, comprising from 1 to 22 carbon atoms (in particular alkyl or alkenyl) and optionally substituted by one or more hydroxyl (—OH) and/or amino (—NRR′) radicals, with R and R′ chosen, independently of one another, from H and C₁-C₆ alkyl; and

R2 and R3 form, with the nitrogen atom to which they are connected, a saturated or unsaturated carbon-comprising heterocycle comprising 5 or 6 ring members, it being possible for one or two nonadjacent carbon atoms optionally to be replaced by an oxygen, sulphur or nitrogen (—NR″) atom, with R″═H or C₁-C₂₂ alkyl, and it being possible for the said heterocycle optionally to be substituted by one or more identical or different radicals chosen from aryl, C₁-C₂₂ alkyl, hydroxyl (—OH) or amino (—NRR′) radicals, with R and R′ chosen, independently of one another, from H and C₁-C₆ alkyl,

R4 denotes:

a linear C₁-C₃₀ or branched C₃-C₃₀ alkyl radical optionally interrupted by a 3-, 4- or 5-membered carbon-comprising ring and/or optionally substituted by (i) an aryl, itself optionally substituted by one or more identical or different radicals chosen from C₁-C₆ alkyl, hydroxyl (—OH) and amino (—NRR′) radicals, with R and R′ chosen, independently of one another, from H and C₁-C₆ alkyl, and/or by (ii) one or more identical or different radicals chosen from hydroxyl (—OH) and amino (—NRR′) radicals, with R and R′ chosen, independently of one another, from H and C₁-C₆ alkyl,

a linear C₂-C₃₀ or branched C₃-C₃₀ alkenyl radical comprising one or more double bonds, in particular from 1 to 6 C═C double bonds, and even from 1 to 3 C═C double bonds, and optionally substituted by one or more identical or different radicals chosen from hydroxyl (—OH) and amino (—NRR′) radicals, with R and R′ chosen, independently of one another, from H and C₁-C₆ alkyl,

an aryl group optionally substituted by one or more identical or different radicals chosen from C₁-C₂₂ alkyl, C₁-C₂₂ alkylcarbonyl, hydroxyl (—OH) and amino (—NRR′) radicals, with R and R′ chosen, independently of one another, from H and C₁-C₆ alkyl;

X⁻ denotes an organic or inorganic anion or a mixture of organic or inorganic anions, in order to ensure the electrical neutrality of the compounds of formula (I).

The carbon-comprising group representing R1, R2 and/or R3 can in particular be a linear C₁-C₂₂ alkyl group, a branched C₃-C₂₂ alkyl group, a linear C₂-C₂₂ alkenyl group or a branched C₃-C₂₂ alkenyl group.

Preferably, n is an integer between 1 and 4, in particular 1, 2 or 3.

Preferably, R1 denotes a linear C₁-C₄ alkyl group, a branched C₃-C₄ alkyl group, a linear C₂-C₄ alkenyl group or a branched C₃-C₄ alkenyl group. Preferably, R1 represents methyl or ethyl.

Preferably, R2 denotes a linear C₁-C₄ alkyl group, a branched C₃-C₄ alkyl group, a linear C₂-C₄ alkenyl group or a branched C₃-C₄ alkenyl group. Preferably, R2 represents methyl or ethyl.

Preferably, R3 denotes a linear C₁-C₂₂ alkyl group, a branched C₃-C₂₂ alkyl group, a linear C₂-C₂₂ alkenyl group or a branched C₃-C₂₂ alkenyl group. Preferably, R3 represents a linear C₁-C₂₂ alkyl group, in particular a linear C₁-C₁₈ alkyl group.

Preferably, R4 denotes:

a linear or branched C₆-C₂₄ alkyl radical, in particular a linear or branched C₈-C₂₂ alkyl radical, it being possible for the said alkyl radical optionally to be interrupted by a 3-, 4- or 5-membered carbon-comprising ring and/or optionally to be substituted by an aryl, itself optionally substituted by one or more identical or different radicals chosen from C₁-C₈ alkyl, hydroxyl and amino radicals;

a linear or branched C₆-C₂₄ alkenyl radical, in particular a linear or branched C₈-C₂₂ alkenyl radical;

an aryl group optionally substituted by one or more identical or different radicals chosen from C₄-C₁₈ alkylcarbonyl (—C(O)—R) and/or hydroxyl radicals.

Preferably, X⁻ denotes an anion or a mixture of anions, such as acetate, lactate, tartrate, citrate, halide, SO₄²⁻, HSO₄⁻, MeSO₄⁻, EtSO₄⁻, mesylate or tosylate, preferably Cl⁻, Br⁻, MeSO₄⁻, EtSO₄⁻, mesylate or tosylate.

The compounds of formula (I) can be used as is or in the form of solvates, in particular of hydrates.

Mention may be made, among the preferred compounds, of the following compounds and their solvates:

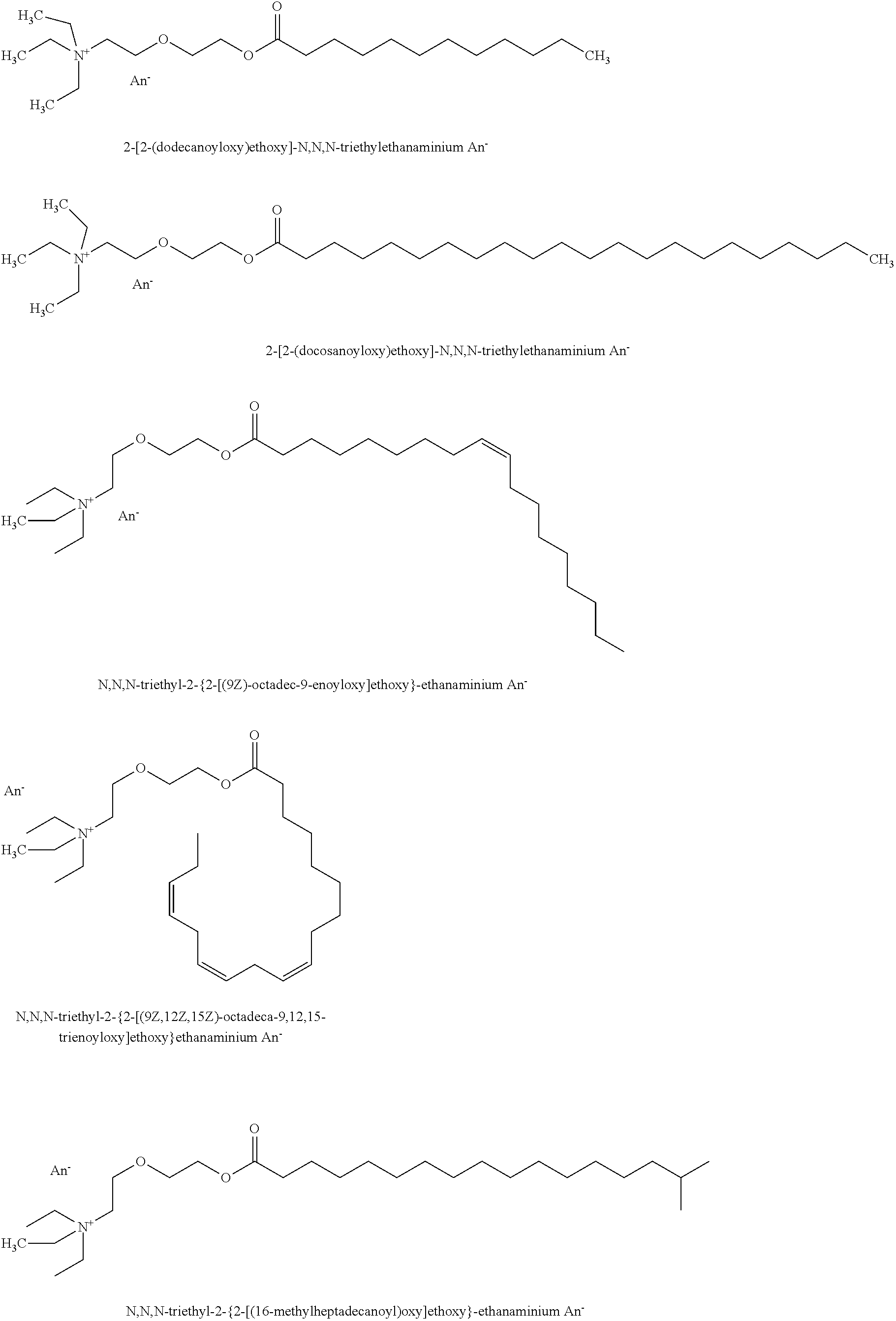

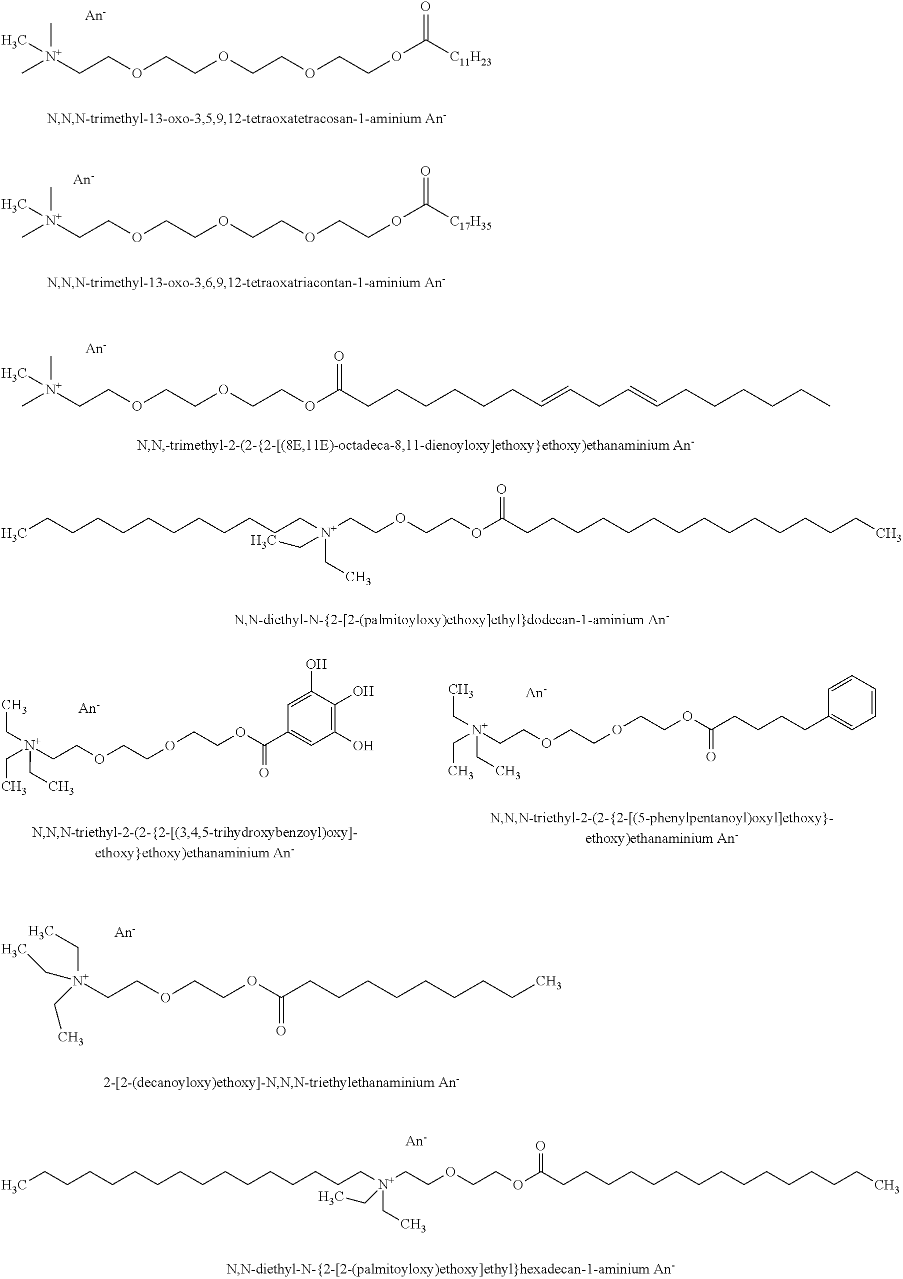

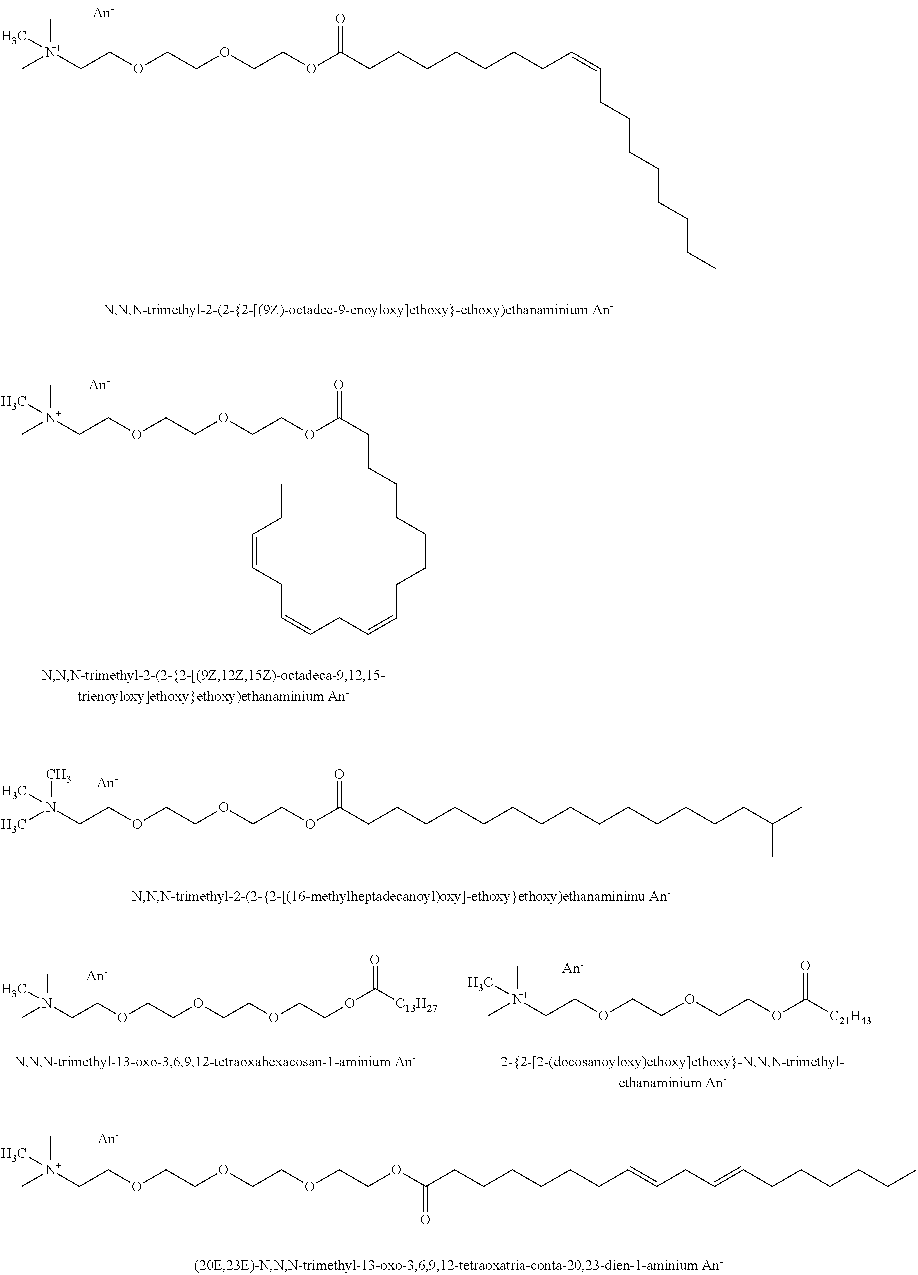

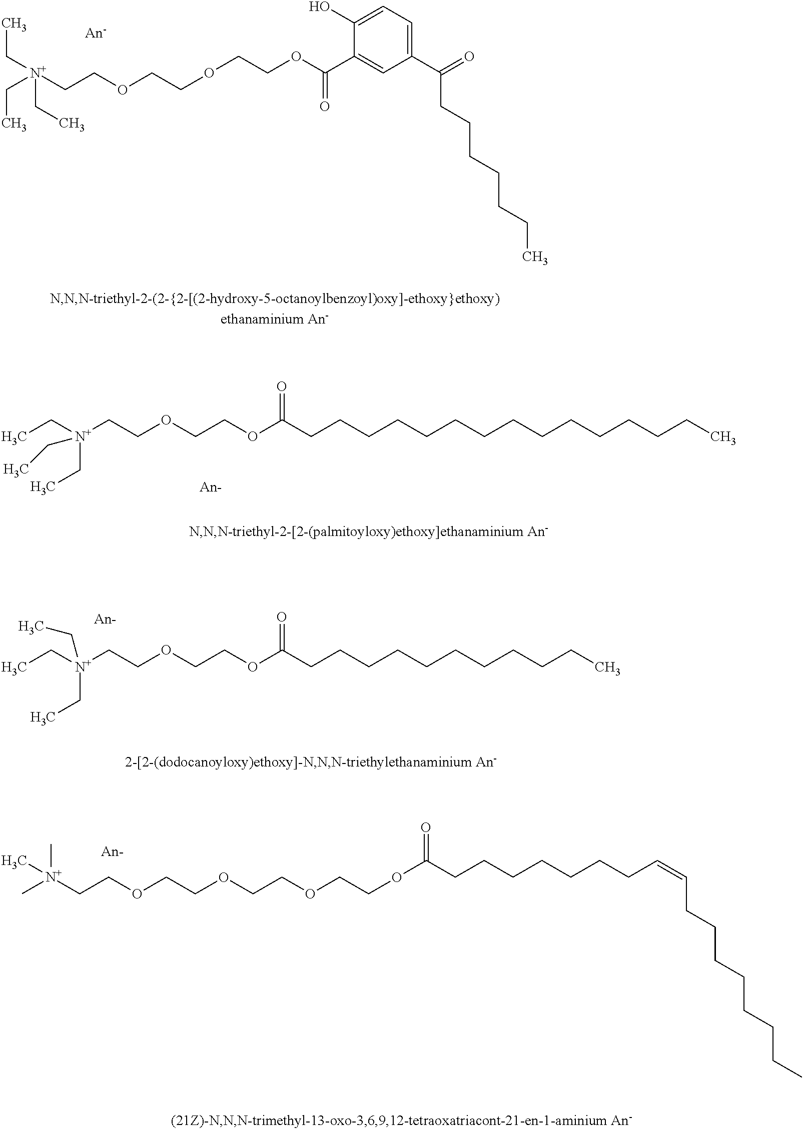

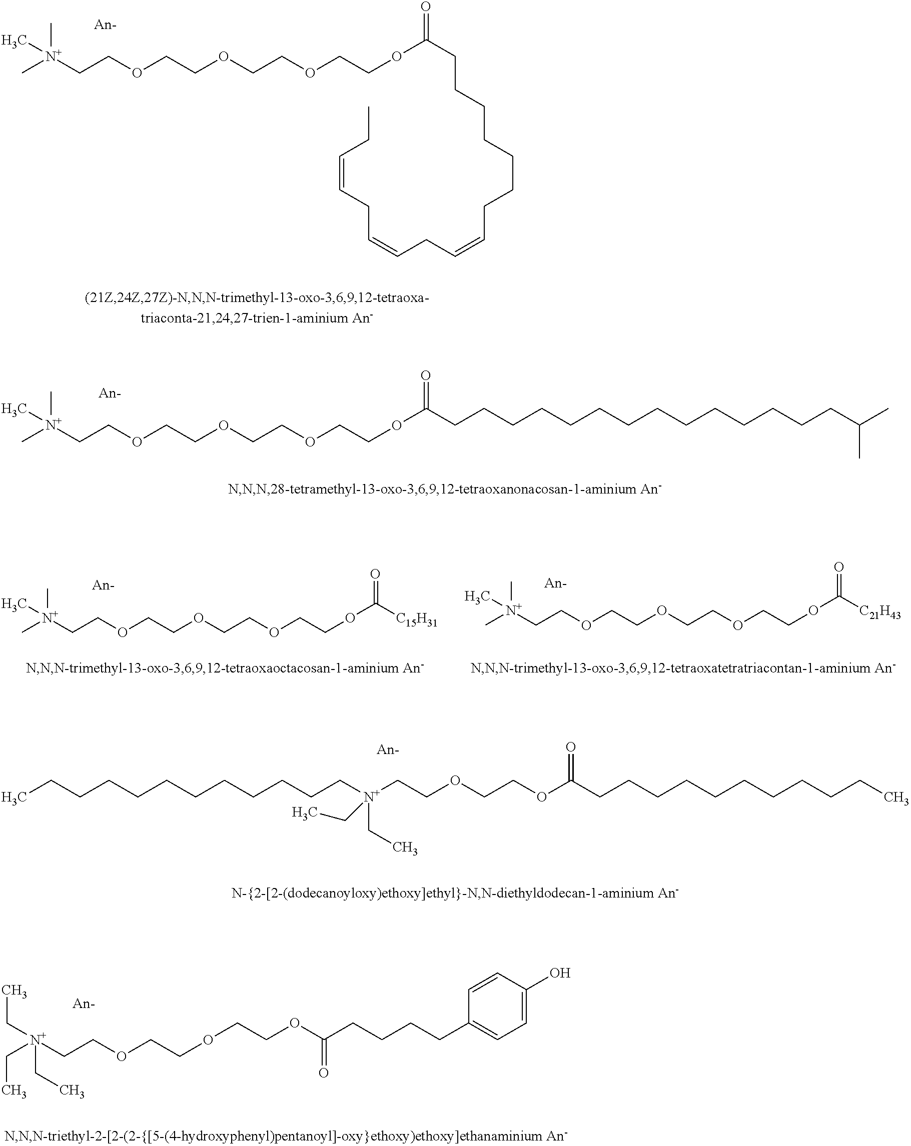

In these compounds, preferably An⁻ (═X⁻) represents Cl⁻, Br⁻, MeSO₄⁻, EtSO₄⁻, SO₄²⁻, HSO₄⁻, mesylate or tosylate, and their mixtures.

Among these compounds, some are novel as such and form the subject-matter of the present invention. They are the compounds of following formula (Ia) and their solvates:

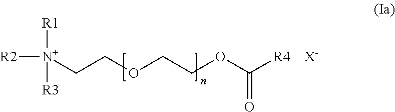

in which:

n is an integer between 1 and 10;

R1=R2=ethyl;

R3 denotes a linear or branched alkyl radical comprising from 1 to 22 carbon atoms which is optionally substituted by one or more hydroxyl (—OH) and/or amino (—NRR′) radicals, with R and R′ chosen, independently of one another, from H and C₁-C₆ alkyl;

R4 denotes:

a linear C₁-C₃₀ or branched C₃-C₃₀ alkyl radical which is optionally interrupted by a 3-, 4- or 5-membered carbon-comprising ring and/or which is optionally substituted by one or more identical or different radicals chosen from hydroxyl (—OH) and amino (—NRR′) radicals, with R and R′ chosen, independently of one another, from H and C₁-C₆ alkyl,

a linear or branched C₆-C₃₀ alkenyl radical comprising one or more double bonds which is optionally substituted by one or more identical or different radicals chosen from hydroxyl (—OH) and amino (—NRR′) radicals, with R and R′ chosen, independently of one another, from H and C₁-C₆ alkyl,

an aryl group which is optionally substituted by one or more identical or different radicals chosen from C₁-C₂₂ alkyl, C₁-C₂₂ alkylcarbonyl, hydroxyl (—OH) and amino (—NRR′) radicals, with R and R′ chosen, independently of one another, from H and C₁-C₆ alkyl; and

X⁻ denotes an organic or inorganic anion or a mixture of organic or inorganic anions, in order to ensure the electrical neutrality of the compounds of formula (Ia).

Preferably, n is an integer between 1 and 4, in particular 1, 2 or 3.

Preferably, R3 represents a linear C₁-C₂₂ alkyl group, in particular a linear C₁-C₁₈ alkyl group.

Preferably, R4 denotes:

a linear or branched C₆-C₂₄ alkyl radical, in particular a linear or branched C₈-C₂₂ alkyl radical, it being possible for the said alkyl radical optionally to be interrupted by a 3-, 4- or 5-membered carbon-comprising ring;

a linear or branched C₆-C₂₄ alkenyl radical, in particular a linear or branched C₈-C₂₂ alkenyl radical;

an aryl group which is optionally substituted by one or more identical or different radicals chosen from C₄-C₁₈ alkylcarbonyl (—C(O)—R) and/or hydroxyl radicals.

Preferably, X⁻ denotes an anion or a mixture of anions, such as acetate, lactate, tartrate, citrate, halide, SO₄²⁻, HSO₄⁻, MeSO₄⁻, EtSO₄⁻, mesylate or tosylate, preferably Cl⁻, Br⁻, MeSO₄⁻ or EtSO₄⁻, and their mixtures.

The compounds according to the invention can be prepared by a person skilled in the art on the basis of his general knowledge.

In particular, they can be prepared according to the following synthesis scheme:

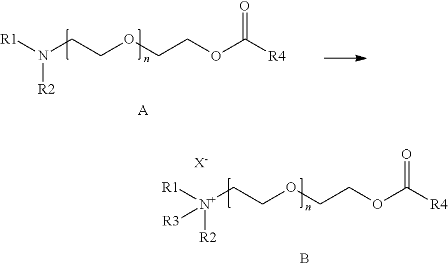

The synthesis of the amines A can in particular be carried out according to the process described in Patent FR 2 869 902.

The quaternized ester B can be obtained by reacting the amine A with an alkylating agent, such as, for example, methyl iodide, dimethyl sulphate, ethyl iodide, diethyl sulphate or a haloalkane, such as a bromohexadecane.

The tertiary amine A and the alkylating agent can be mixed and heated at 15° C.-140° C. for 2 to 80 hours. After cooling, the excess alkylating agent can be removed by washing operations with diisopropyl ether.

The solid obtained can be filtered off, preferably under an inert atmosphere, washed and dried under reduced pressure, optionally in the presence of P₂O₅.

An ion exchange can be carried out on conclusion of the reaction, by contact with an ion-exchange resin chosen according to the exchanges desired. These resins are, for example, IRA 402 (alkyl sulphates to chlorides exchange) or IRA 400 (iodide to chloride exchange). Thus, the anion can be exchanged by treatment of an aqueous or (aqueous)alcoholic solution of the compound with the ion-exchange resin. The solvent can be removed and the product can be washed, for example with diisopropyl ether, and then filtered off and dried under reduced pressure, optionally in the presence of P₂O₅.

The compounds according to the invention, (I) or (Ia), have a very particular application in the cosmetic or pharmaceutical field, in particular in the hair field, especially as conditioning agent.

The amount of compound (I) or (Ia), alone or as a mixture, present in the compositions depends, of course, on the type of composition and on the properties desired and can vary within a very broad range generally of between 0.01 and 50% by weight, preferably between 0.1 and 30% by weight, in particular between 0.5 and 25% by weight, indeed even between 1 and 20% by weight, better still between 1.5 and 10% by weight, with respect to the total weight of the composition.

The compositions according to the invention can very obviously comprise a mixture of compounds of formula (I) or (Ia).

The compositions according to the invention can be provided in all the formulation forms conventionally used and in particular in the form of an aqueous, alcoholic, aqueous/alcoholic or oily solution or suspension, of a solution or dispersion of the lotion or serum type, of an emulsion, in particular with a liquid or semiliquid consistency, of the 0/W, W/O or multiple type, of a suspension or emulsion with a soft consistency of (O/W) or (W/O) cream type, of an aqueous or anhydrous gel, or of any other cosmetic form.

These compositions can be packaged in pump-action sprays or in aerosol containers, in order to provide for application of the composition in the vaporized form (lacquer) or in the foam form. Such packaging forms are indicated, for example, when it is desired to obtain a spray or a foam, for the treatment of the hair. In these cases, the composition preferably comprises at least one propellant.

Preferably, the composition is provided in the form of an emulsion comprising the compound of formula (I) or (Ia) in dispersion in an aqueous phase or else in solution in a fatty phase.

The compositions according to the invention comprise a physiologically acceptable medium, that is to say a medium compatible with keratinous substances, in particular the skin of the face or of the body, the lips, the hair, the eyelashes, the eyebrows and the nails. The said physiologically acceptable medium is preferably a cosmetically acceptable medium, the composition then being a cosmetic composition intended in particular for a topical application.

The said physiologically acceptable medium preferably comprises at least one standard cosmetic ingredient chosen in particular from propellants, water, carbon-comprising oils, C₈-C₄₀ esters, C₈-C₄₀ acids, C₁-C₄₀ alcohols, organic solvents, nonionic surfactants, cationic surfactants, anionic surfactants, amphoteric surfactants, zwitterionic surfactants, sunscreens, moisturizing agents, antidandruff agents, antioxidants, reducing agents, oxidation bases, couplers, oxidizing agents, direct dyes, hair-straightening agents, pearlescent and opacifying agents, plasticizing or coalescence agents, hydroxy acids, pigments, fillers, silicones, polymeric or nonpolymeric thickeners, emulsifiers or polymers, in particular conditioning or styling polymers. The said medium can very obviously comprise several cosmetic ingredients appearing in the above list.

According to their nature and the destination of the composition, the standard cosmetic ingredients can be present in standard amounts which can be easily determined by a person skilled in the art and which can be, for each ingredient, between 0.01 and 80% by weight.

The composition can in particular comprise water and/or one or more C₁-C₄₀ alcohols; mention may in particular be made of C₁-C₇ aliphatic or aromatic mono-alcohols, polyols and polyol ethers, which can thus be employed alone or as a mixture with water; advantageously, the composition comprises a water/ethanol, water/isopropanol or water/benzylalcohol mixture.

The oils, in particular carbon-comprising oils, indeed even hydrocarbon oils, can be present in a proportion of 0.01 to 20% by weight, in particular of 0.02 to 10% by weight, with respect to the total weight of the composition. Mention may in particular be made of vegetable, animal or mineral oils, which are or are not hydrogenated, saturated or unsaturated, linear or branched and cyclic or aliphatic hydrocarbon synthetic oils, such as, for example, poly-α-olefins, in particular polydecenes and polyisobutenes, volatile or nonvolatile, organomodified or nonorganomodified and water-soluble or water-insoluble silicone oils, fluorinated or perfluorinated oils, and their mixtures.

The alcohols, the esters and the acids having from 8 to 40 carbon atoms can be present in a proportion of 0.01 to 50% by weight, in particular of 0.1 to 20% by weight, with respect to the total weight of the composition.

Mention may in particular be made of fatty alcohols possessing linear or branched C₁₂-C₃₂, in particular C₁₂-C₂₆, chains and especially of cetyl alcohol, stearyl alcohol, cetearyl alcohol, isostearyl alcohol, octyldodecanol, 2-butyloctanol, 2-hexyldecanol, 2-undecylpentadecanol, oleyl alcohol or linoleyl alcohol.

Mention may also be made of oxyalkylenated, in particular oxyethylenated, C₈-C₄₀, in particular C₁₆-C₂₀, fatty alcohols, preferably comprising from 10 to 50 mol of ethylene oxide and/or propylene oxide, such as oleth-12, ceteareth-12 and ceteareth-20, oxypropylenated stearyl alcohol comprising in particular 15 mol of propylene oxide, oxyethylenated lauryl alcohol comprising in particular more than 7 oxyethylene groups, and their mixtures.

Mention may also be made of fatty acids possessing linear or branched C₁₆-C₄₀ chains and in particular of 18-methyleicosanoic acid, coconut oil or hydrogenated coconut oil acids, stearic acid, lauric acid, palmitic acid, oleic acid, behenic acid and their mixtures.

Mention may also be made of fatty esters possessing linear or branched chains and comprising in total between 8 and 40 carbon atoms, such as esters of monoalcohols or of polyols of fatty acids comprising from 8 to 30 carbon atoms, and their oxyalkylenated and in particular oxyethylenated derivatives, the polyols being preferably chosen from sugars, C₂-C₆ alkylene glycols, glycerol, polyglycerols, sorbitol, sorbitan, polyethylene glycols, polypropylene glycols and their mixtures. Mention may be made, as esters of monoalcohols, of isopropyl myristate or palmitate, and also myristyl, cetyl and stearyl myristates, palmitates and stearates, alone or as a mixture.

The composition can also comprise, as carbon-comprising oils, vegetable oils, such as avocado oil, olive oil, apricot oil, argan oil, jojoba oil or shea butter, which can be present in a proportion of 0.1 to 10% by weight in the composition, in particular of 0.2 to 5% by weight.

In a preferred embodiment, the composition according to the invention comprises C₈-C₄₀ fatty alcohols which can be present very particularly in an amount of 1 to 15% by weight, in particular of 2.5 to 10% by weight, indeed even of 3 to 8% by weight, in the cosmetic composition.

These fatty alcohols can be saturated or unsaturated and linear or branched; mention may in particular be made of cetyl alcohol, stearyl alcohol, isostearyl alcohol, oleyl alcohol, ricinoleyl alcohol or linoleyl alcohol, alone or as a mixture; saturated fatty alcohols, alone or as a mixture, are preferred.

The nonionic, cationic, anionic, amphoteric or zwitterionic surfactants, other than those of formula (I), and their mixtures can be present in a proportion of 0.01 to 50% by weight, in particular of 0.1 to 40% by weight, indeed even of 0.5 to 30% by weight, better still of 1 to 15% by weight, with respect to the total weight of the composition.

In particular, the ratio by weight of the amount of fatty alcohols to the amount of surfactants in the composition is preferably greater than or equal to 1.5, in particular between 1.5 and 10, indeed even between 1.6 and 8, better still between 1.7 and 6.

The propellants can be present in a proportion of 5 to 90% by weight, with respect to the total weight of the composition, more particularly in a proportion of 10 to 60% by weight.

The sunscreens can be present in a proportion of 0.01 to 20% by weight, in particular of 0.5 to 10% by weight, with respect to the total weight of the composition.

The moisturizing agents can be present in a proportion of 0.01 to 20% by weight, in particular of 0.1 to 7% by weight, with respect to the total weight of the composition.

The antidandruff agents can be present in a proportion of 0.001 to 20% by weight, in particular of 0.01 to 10% by weight, with respect to the total weight of the composition, preferably of 0.1 to 5% by weight.

The antioxidants can be present in a proportion of 0.05 to 1.5% by weight, with respect to the total weight of the composition.

The reducing agents can be present in a proportion of 0.1 to 30% by weight, in particular of 0.5 to 20% by weight, with respect to the total weight of the composition.

The oxidation bases can be present in an amount of between 0.001 and 10% by weight, preferably of 0.005 to 6% by weight, of the total weight of the composition.

The couplers can be present in an amount of between 0.001 and 10% by weight, preferably of 0.005 to 6% by weight, of the total weight of the composition.

The oxidizing agents can be present in an amount of between 1 and 40% by weight, preferably of between 1 and 20% by weight, with respect to the weight of the composition.

The direct dyes can be present in an amount of between 0.001 and 20% by weight, preferably of 0.01 to 10% by weight, with respect to the total weight of the composition.

The hair-straightening agents can be present in a proportion of 0.01 to 3.5% by weight, in particular of 0.05 to 1.5% by weight, with respect to the total weight of the composition.

The pearlescent and opacifying agents can be present in a proportion of 0.01 to 3% by weight, in particular of 0.05 to 2.5% by weight, with respect to the total weight of the composition.

The plasticizing or coalescence agents can be present in a proportion of 0.1 to 25% by weight, in particular of 1 to 10% by weight, with respect to the total weight of the composition.

The hydroxy acids can be present in a proportion of 1 to 10% by weight, in particular of 2 to 5% by weight, with respect to the total weight of the composition.

The pigments and fillers can be present in a proportion of 0.01 to 50% by weight, in particular of 0.02 to 30% by weight, with respect to the total weight of the composition.

The silicones can be volatile or nonvolatile; mention may in particular be made of polyorganosiloxanes which are or are not modified, namely polyorganosiloxane oils, gums and resins, as is or in the form of solutions in organic solvents or in the form of emulsions or microemulsions. They can be present in an amount of 0.01 to 40% by weight, in particular of 0.05 to 20% by weight, with respect to the total weight of the composition.

The polymeric or nonpolymeric thickeners can be present in a proportion of 0.01 to 10% by weight, in particular of 0.1 to 5% by weight, with respect to the total weight of the composition.

The polymers, in particular conditioning or styling polymers, and in particular polymers which are soluble in water or soluble in carbon-comprising and/or silicone oils, can be present in a proportion of 0.01 to 20% by weight, in particular of 0.1 to 10% by weight, with respect to the total weight of the composition.

A person skilled in the art will take care to choose the ingredients participating in the composition, and their amounts, so that they do not interfere with the properties of the compositions of the present invention.

The cosmetic composition according to the invention can be provided in the form of a product for caring for, cleaning and/or making up the skin of the body or of the face, the lips, the eyebrows, the eyelashes, the nails and the hair, of an antisun or self-tanning product, of a body hygiene product or of a hair product, in particular for caring for, cleaning, styling, shaping or dyeing the hair.

In particular, it has a particularly advantageous application in the hair field, in particular for the form retention of the hairstyle or the shaping of the hair, or also the care, cosmetic treatment or cleaning of the hair. The hair compositions are preferably shampoos, hair conditioners, styling or care gels, care lotions or creams, conditioners, hair setting lotions, blow drying lotions, fixing and styling compositions, such as lacquers or sprays, hair restructuring lotions, lotions or gels for combating hair loss, antiparasitic shampoos, antidandruff lotions or shampoos or shampoos for treating seborrhoea. The lotions can be packaged in various forms, in particular in vaporizers, pump-action sprays or aerosol containers, in order to provide for application of the composition in the vaporized form or in the foam form.

It can also be provided in the form of a hair dyeing product, in particular oxidation dyeing or direct dyeing product, optionally in the form of a colouring shampoo, in the form of a perming, hair straightening or bleaching composition, or also in the form of a rinse-out composition, to be applied before or after a dyeing, bleaching, perming or hair straightening operation or also between the two stages of a perming operation or hair straightening operation.

The composition according to the invention can also be provided in the form of a care composition, in particular a moisturizing composition, for the skin of the body or of the face, the lips and/or the superficial body growths, in particular of a care product intended to cosmetically treat the skin and in particular to moisturize it, to smooth it, to depigment it, to nourish it, to protect it from sunlight or to confer a specific cosmetic treatment on it. Thus, it can be a lip care base, a fixing base for lipsticks, an antisun protection or artificial tanning composition, a care (day, night, antiageing or moisturizing) composition for the face, a matifying composition, a composition for cleaning the skin, for example a make-up-removing product or a bath or shower gel or a cleaning bar or soap, a body hygiene composition, in particular a deodorant or antiperspirant product, or also a depilatory composition, an aftershave gel or an aftershave lotion.

It can also be provided in the form of a product for making up the skin of the body or of the face, the lips, the eyelashes, the nails or the hair, in particular a foundation, a blusher, a face powder, an eyeshadow, a concealer, an eyeliner, a mascara, a lipstick, a lip gloss, a lip pencil, a nail varnish, a nail care product or a product for the temporary tattooing of the skin of the body.

More particularly still, the composition according to the invention has an advantageous application in the care and the cosmetic treatment, in particular the protection, of the hair, especially weakened and/or damaged hair, for example hair weakened and/or damaged by chemical or mechanical treatments; use may in particular be made of the compounds according to the invention in post-treatment, after a stage of dyeing, bleaching or straightening the hair.

A subject-matter of the invention is thus a method for the cosmetic treatment, in particular for the making up, the care, the cleaning, the dyeing or the shaping, of keratinous substances, in particular of the skin of the body or of the face, the lips, the nails, the hair and/or the eyelashes, comprising the application, to the said substances, of a cosmetic composition comprising at least one compound according to the invention.

Preferably, it is a cosmetic treatment method for the conditioning of the hair, in particular for providing it with suppleness, disentangling, smoothing and/or the ability to be combed or for improving the suppleness, the disentangling, the smoothing and/or the ability to be combed thereof.

The application of the composition can optionally be followed by a rinsing stage and/or optionally by a heat treatment stage.

The invention is illustrated in more detail in the following examples.

### EXAMPLE 1

### Synthesis of 2-[2-(dodecanoyloxy)ethoxy]-N,N,N-triethylethanaminium chloride

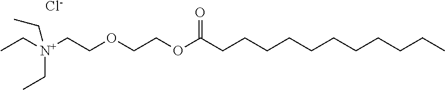

4 g (1 eq.) of 2-[2-(diethylamino)ethoxy]ethyl laurate and 10 ml (10.8 eq.) of ethyl iodide are mixed; mixing is carried out at 25° C. for 72 hours.

The excess alkylating agent is subsequently removed by washing with diisopropyl ether. The solid obtained is then filtered off under nitrogen, washed and dried under reduced pressure in the presence of P₂O₅. The iodide anion is exchanged by treatment of an aqueous solution of the compound with 50 g of ion-exchange resin IRA 400.

After removing the solvent, washing the compound with diisopropyl ether, filtering and drying under reduced pressure in the presence of P₂O₅, 2.8 g of the desired product are obtained, which product is provided in the form of a beige powder (yield: 59%).

M (m/z): M⁺=372

### EXAMPLE 2

### Synthesis of 2-[2-(decanoyloxy)ethoxy]-N,N,N-triethylethanaminium chloride

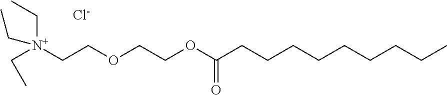

8 g (1 eq.) of 2-[2-(diethylamino)ethoxy]ethyl decanoate and 6.6 ml (2 eq.) of diethyl sulphate are mixed; mixing is carried out for 7 hours while heating at 60° C.

The excess alkylating agent is subsequently removed by washing with diisopropyl ether. The solid obtained is then filtered off under nitrogen, washed and dried under reduced pressure in the presence of P₂O₅. The anion is exchanged by treatment of an aqueous solution of the compound with 300 g of ion-exchange resin IRA 402.

After removing the solvent, washing the compound with diisopropyl ether, filtering and drying under reduced pressure in the presence of P₂O₅, 5.6 g of the desired product are obtained, which product is provided in the form of a white powder (yield: 87%).

M (m/z): M⁺=344

### EXAMPLE 3

### Synthesis of N,N,N-triethyl-2-[2-(palmitoyl-oxy)ethoxy]ethanaminium chloride

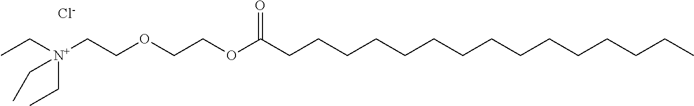

7.7 g (1 eq.) of 2-[2-(diethylamino)ethoxy]ethyl palmitate and 5.5 ml (2 eq.) of diethyl sulphate are mixed; mixing is carried out for 15 hours while heating at 65-70° C.

The excess alkylating agent is subsequently removed by washing with diisopropyl ether. The solid obtained is then filtered off under nitrogen, washed and dried under reduced pressure in the presence of P₂O₅. The iodide anion is exchanged by treatment of an aqueous solution of the compound with 300 g of ion-exchange resin IRA 402.

After removing the solvent, washing the compound with diisopropyl ether, filtering and drying under reduced pressure in the presence of P₂O₅, 7.15 g of the desired product are obtained, which product is provided in the form of a white powder (yield: 80%).

M (m/z): M⁺=428

### EXAMPLE 4

### Synthesis of 2-[2-(docosanoyloxy)ethoxy]-N,N,N-triethylethanaminium chloride

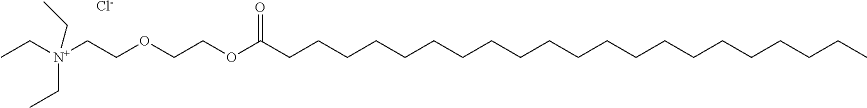

5.8 g (1 eq.) of 2-[2-(diethylamino)ethoxy]ethyl docosanoate and 3.15 ml (2 eq.) of diethyl sulphate are mixed; mixing is carried out for 15 hours while heating at 65-70° C.

The excess alkylating agent is subsequently removed by washing with diisopropyl ether. The solid obtained is then filtered off under nitrogen, washed and dried under reduced pressure in the presence of P₂O₅. The iodide anion is exchanged by treatment of the compound, dissolved in a pure water/acetone (80/20) solution, on 300 g of ion-exchange resin IRA 402. The compound is eluted with a pure water/acetone (80/20) mixture.

After removing the solvent, washing the compound with diisopropyl ether, filtering and drying under reduced pressure in the presence of P₂O₅, 5.5 g of the desired product are obtained, which product is provided in the form of a white powder (yield: 84%).

M (m/z): M⁺=512

### EXAMPLE 5

### Synthesis of N,N-diethyl-N-{2-[2-(palmitoyl-oxy)ethoxy]ethyl}hexadecan-1-aminium bromide

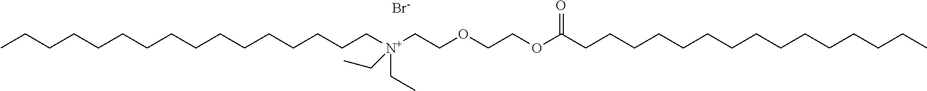

5 g (1 eq.) of 2-[2-(diethylamino)ethoxy]ethyl palmitate and 7.63 ml (2 eq.) of bromohexane are mixed; mixing is carried out for 48 hours while heating at 120° C.

The excess alkylating agent is subsequently removed by washing with diisopropyl ether. The solid obtained is then filtered off under nitrogen, washed with diisopropyl ether and dried under reduced pressure in the presence of P₂O₅.

4 g of the desired product are obtained, which product is provided in the form of a beige powder (yield: 76%).

M (m/z): M⁺=625

### EXAMPLE 6

### Synthesis of the Following Compound

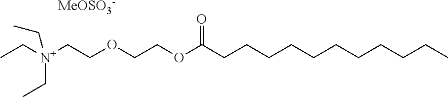

30 ml of diethyl sulphate (2 eq.) are run dropwise onto 38.6 g of the corresponding tertiary amine. The reaction medium is left stirring under an inert atmosphere (nitrogen) at 55° C. for 20 hours. The reaction medium is then washed 3 times with diisopropyl ether, in order to remove the excess diethyl sulphate.

The product obtained is then purified on a sintered silica funnel.

Eluent: CH₂Cl₂/MeOH (98/2), an eluent gradient is subsequently produced.

38 g of the desired product are obtained, which product is provided in the form of a light brown paste (yield: 71%).

M (m/z): M⁺=372

### EXAMPLE 7

The following hair cosmetic composition is prepared (% by weight):

| | |
|---|---|
| Chicory root inulin (Inutec N25 from Orafti) | 15% AM |
| Lemon pectin (CU 201 from Herbstreith and Fox) | 2% AM |
| Compound of Example 2 | 1% |
| Water | q.s. for 100% |

1 g of the composition is applied to a lock weighing 2.5 g of bleached hair (SA20) wetted beforehand. The lock is subsequently rinsed and combed. It is observed that the wet lock has a smooth feel and that it is supple and easy to disentangle.

The lock is dried under a hood dryer for 15 minutes. The dry lock is, to the touch, soft and smooth up to the ends. It disentangles well.

Similarly, hair compositions can be prepared with the compounds of Examples 1 and 3 to 6.

### EXAMPLE 8

The following hair cosmetic composition is prepared (% by weight):

| | |
|---|---|
| Cetearyl alcohol | 7% AM |
| Cetyl esters | 1.5% AM |
| Compound of Example 3 | 3% |
| Water | q.s. for 100% |

A comparative composition is prepared by replacing the compound according to the invention with 2-(2-(diethylamino)ethoxy)ethyl palmitate (3% by weight).

After storing the compositions obtained at 25° C. for 8 hours, they are observed visually and it is found that the composition according to the invention is stable (homogeneous), whereas the comparative composition is not stable: phase separation is observed.

### EXAMPLE 9

Synthesis of a compound of formula (I) in which n=1, R4=C₂₁H₄₃/R1=R2=R3=ethyl and X⁻ is a mixture of ethosulphate and hydrogensulphate.

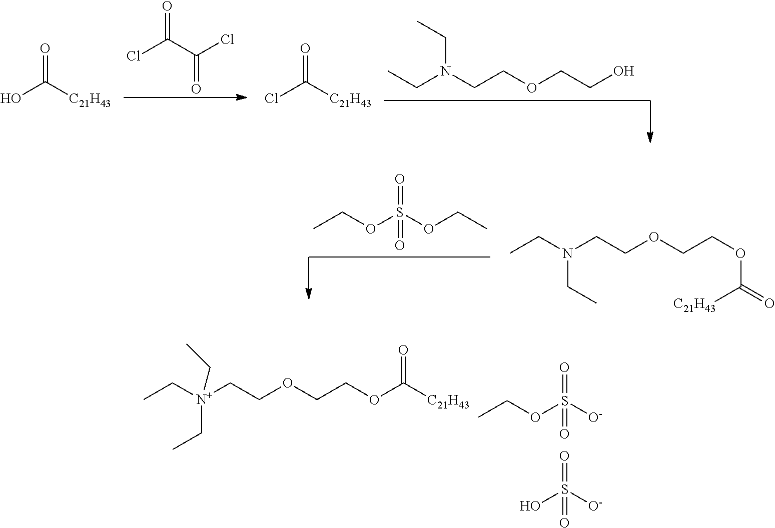

### Stage 1: Synthesis of 2-[2-(diethylamino)ethoxy]ethyl docosanoate of formula

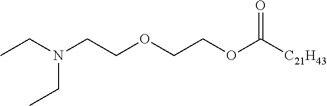

45 ml of triethylamine (0.32 mol) were added to a solution of 25 g of 2-[2-(diethylamino)ethoxy]ethanol (0.155 mol) dissolved in 500 ml of dichloromethane. After stirring at ambient temperature for 30 minutes, a solution of 55 g of the acid chloride of behenic acid was added dropwise. A slight exotherm of 8° C. occurs. On completion of the addition, the reaction mixture was kept stirred for 8 hours. After filtering through a sintered glass funnel, the reaction medium was poured onto 500 ml of water and then the organic phase was washed with water until a pH of 9 was achieved. After drying over sodium sulphate and filtering, the organic phase was evaporated under reduced pressure in order to obtain 70 g of the expected compound in the form of a white paste with a yield of 93%.

Melting point: 88.7° C.

¹H NMR spectrum 400 MHz and ¹³C NMR spectrum 100 MHz: in accordance

Mass spectrum in accordance: M (m/z): [M+H]⁺=484

### Stage 2: Synthesis of the 2-[2-(docosanoyloxy)ethoxy]-N,N,N-triethylethanaminium ethosulphate/hydrogen-sulphate mixture

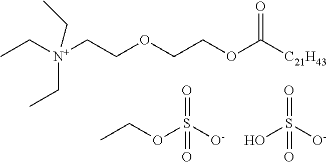

41 ml of diethyl sulphate (0.312 mol) were added, all at once, to 65 g of 2-[2-(diethylamino)ethoxy]ethyl docosanoate (0.134 mol) introduced into a reactor. The reaction medium was subsequently brought to 40° C. with stirring for 48 hours. 50 ml of acetone were subsequently added and stirring was maintained until the reaction medium had dissolved. This medium was subsequently poured onto 2 litres of isopropyl ether kept stirred and under argon. The precipitate formed was subsequently filtered off on a sintered glass funnel and then dried under vacuum in order to obtain 71 g of 2-[2-(docosanoyloxy)ethoxy]-N,N,N-triethylethanaminium in the form of an ethosulphate/hydrogensulphate mixture, the percentage of which is determined by NMR: 0.52/0.48. The percentage of hydrogensulphate can vary according to the content of water present in the precipitation medium.

The product is provided in the form of a white powder; the yield is 83%.

Melting point: 76.3° C.

¹H NMR spectrum 400 MHz and ¹³C NMR spectrum 100 MHz: in accordance

Mass spectrum in accordance: M (m/z): [M]⁺=512; [M]⁻=97 and 125

### EXAMPLE 10

Synthesis of a compound of formula (I) in which n=1, R4=C₂₁H₄₃, R1=R2=R3=ethyl and X⁻ is hydrogensulphate.

0.1 ml of concentrated 36% sulphuric acid was added to a suspension of 2 g of the 2-[2-(docosanoyl-oxy)ethoxy]-N,N,N-triethylethanaminium hydrogen-sulphate/ethosulphate mixture prepared in Example 9 in 100 ml of acetone. The reaction mixture was subsequently brought to reflux for 5 hours and then, after cooling to 5° C., a precipitate was formed. After filtering off and drying under vacuum, 1.2 g of the expected product were thus obtained entirely in the form of hydrogensulphate.

¹H NMR spectrum 400 MHz and ¹³C NMR spectrum 100 MHz: in accordance

Mass spectrum in accordance: M (m/z): [M]⁺=512; [M]⁻=97

### EXAMPLE 11

Synthesis of a compound of formula (I) in which n=1, R4=C₂₁H₄₃, R1=R2=R3=ethyl and X⁻ is methanesulphonate (mesylate).

### General Synthesis Route for Examples 11 and 12:

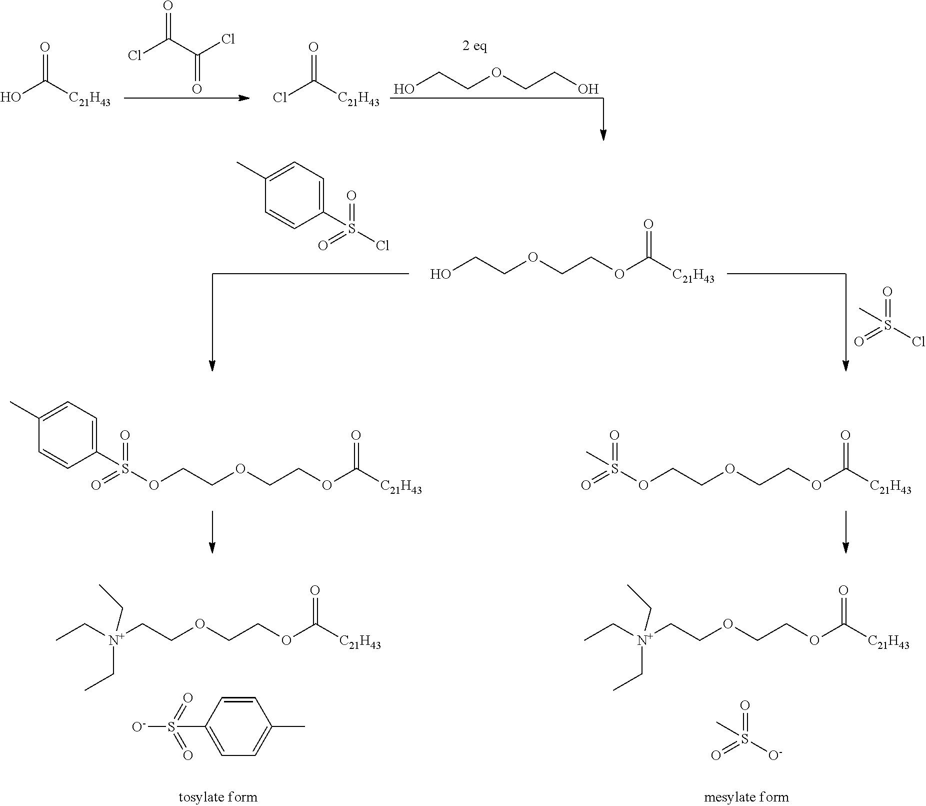

### Stage 1: Synthesis of 2-(2-hydroxyethoxy)ethyl docosanoate

250 ml of diethylene glycol (2.6 mol) were added to a solution of 250 ml of dichloromethane and then the reaction medium was cooled to 5° C. with an ice bath. 45 g of N,N-diisopropylethylamine (0.348 mol) were subsequently added and the mixture was kept stirred for 30 minutes. A solution of 84 g (0.233 mol) of the acid chloride of behenic acid, dissolved beforehand in 250 ml of dichloromethane, was subsequently added dropwise so as to maintain a temperature of between 5° C. and 10° C. On completion of the addition, the reaction medium was kept stirred at ambient temperature for 6 hours. The reaction medium was subsequently run onto 2 litres of water and then the organic phase was washed three times with 300 ml of water and then, finally, with 200 ml of a saturated ammonium chloride solution. After drying over sodium sulphate and filtering, the organic phase was evaporated under reduced pressure; 92 g of the expected compound were obtained in the form of a white powder, with a yield of 91%.

¹H NMR spectrum 400 MHz and ¹³C NMR spectrum 100 MHz: in accordance

Mass spectrum: M (m/z): [M+H]⁺=429

### Stage 2: Synthesis of 2-{2-[(methylsulphonyl)oxy]-ethoxy}ethyl docosanoate

1.7 g of N,N-diisopropylethylamine (0.013 mol) were added to a solution, cooled to 5° C., of 5 g of 2-(2-hydroxyethoxy)ethyl docosanoate (0.0116 mol) dissolved in 100 ml of dichloromethane. A solution of 1.33 g of methanesulphonic acid chloride (0.0166 mol) dissolved in 50 ml of dichloromethane were subsequently added dropwise, so as to maintain a temperature of less than 10° C. On completion of the addition, the reaction mixture was kept stirred at ambient temperature for 5 hours. The reaction mixture was subsequently poured onto 300 ml of water and the organic phase was washed twice with 150 ml of water and then once with 100 ml of a saturated ammonium chloride solution. After drying over sodium sulphate, filtering and evaporating under reduced pressure, the crude product obtained was purified on a silica column in a dichloro-methane/methanol mixture.

3.5 g of the expected compound were obtained in the form of a white powder, with a yield of 60%.

¹H NMR spectrum 400 MHz and ¹³C NMR spectrum 100 MHz: in accordance

Mass spectrum: M (m/z): [M+Na]⁺=529

### Stage 3: Synthesis of 2-[2-(docosanoyloxy)ethoxy]-N,N,N-triethylethanaminium methanesulphonate

25 ml of triethylamine (0.179 mol) were added to a suspension of 3.4 g of 2-{2-[(methylsulphonyl)oxy]-ethoxy}ethyl docosanoate (0.00691 mol) in 100 ml of acetonitrile. The reaction medium was subsequently brought to reflux for 72 hours. After evaporating under reduced pressure, the residue was taken up in 100 ml of acetone and the precipitate formed was filtered off.

After drying under reduced pressure, 3 g of the expected compound were obtained in the form of a white powder, with a yield of 71%.

¹H NMR spectrum 400 MHz and ¹³C NMR spectrum 100 MHz: in accordance

Mass spectrum: M (m/z): [M]⁺=512; [M]⁻=95

### EXAMPLE 12

Synthesis of a compound of formula (I) in which n=1, R4=C₂₁H₄₃, R1=R2=R3=ethyl and X⁻ is para-toluenesulphonate (tosylate).

### Stage 1: Synthesis of 2-(2-{[(4-methylphenyl)sulphonyl]oxy}ethoxy)ethyl docosanoate

17 g of triethylamine (0.168 mol) were added to a solution, cooled to 5° C., of 70 g of 2-(2-hydroxy-ethoxy)ethyl docosanoate (0.1633 mol) dissolved in 500 ml of dichloromethane. A solution of 32 g of para-toluenesulphonic acid chloride (0.1678 mol) dissolved in 250 ml of dichloromethane was subsequently added dropwise, so as to maintain a temperature of less than 10° C. On completion of the addition, the reaction mixture was kept stirred at ambient temperature for 12 hours. The reaction mixture was subsequently run onto 2 litres of water and the organic phase was washed with 3 times 300 ml of water, then 3 times 200 ml of a saturated sodium hydrogencarbonate solution and 1 times 200 ml of a saturated ammonium chloride solution. After drying over sodium sulphate, filtering and evaporating under reduced pressure, the crude compound obtained was purified on a silica column in a dichloromethane/methanol mixture. 70 g of the expected compound were thus obtained in the form of a white powder, with a yield of 74%.

¹H NMR spectrum 400 MHz and ¹³C NMR spectrum 100 MHz: in accordance

Mass spectrum: M (m/z): [M+Na]⁺=605

### Stage 2: Synthesis of 2-[2-(docosanoyloxy)ethoxy]-N,N,N-triethylethanaminium 4-methylbenzenesulphonate

25 ml of triethylamine (0.179 mol) were added to a suspension of 10.66 g of 2-(2-{[(4-methylphenyl)-sulphonyl]oxy}ethoxy)ethyl docosanoate (0.0182 mol) in 100 ml of acetonitrile. The reaction medium was subsequently brought to reflux for 36 hours. After evaporating under reduced pressure, the residue was taken up in 100 ml of acetone and the precipitate formed was filtered off. After drying under reduced pressure, 11 g of the expected compound were obtained in the form of a white powder, with a yield of 88%.

¹H NMR spectrum 400 MHz and ¹³C NMR spectrum 100 MHz: in accordance

Mass spectrum: M (m/z): [M]⁺=512; [M]⁻=171

### EXAMPLE 13

Various cosmetic compositions are prepared comprising the compounds prepared in Examples 9 to 12 (% by weight).

Some of these compositions are applied to the hair in the following way: 1 g of cosmetic composition is applied to a bleached lock (SA20) of hair weighing 2.5 g.

### Composition 1:

| | |
|---|---|
| Compound of Example 9 or of Example 12 | 1.5% |
| Cetearyl alcohol (C₁₆/C₁₈ 50/50) | 3% |
| (Lanette O gold flakes from Cognis) | |
| Citric acid | q.s. pH 2.5 |
| Water | q.s. for 100% |

These compositions contribute a smooth feel to wet hair and the lock is rendered supple and easily disentangles. On dry hair, the lock disentangles easily and the feel is smooth.

Similar cosmetic compositions can be prepared comprising the compounds of Examples 10 and 11.

### Composition 2:

| | |
|---|---|
| Compound of Example 9 | 1.5% |
| Cetearyl alcohol (C₁₆/C₁₈ 50/50) | 3% |
| (Lanette O gold flakes from Cognis) | |
| Myristyl/cetyl/stearyl | 0.5% |
| myristate/palmitate/stearate mixture | |
| (Crodamol MS-V from Croda) | |
| Citric acid | q.s. pH 2.5 |
| Water | q.s. for 100% |

This composition contributes a smooth feel to wet hair and the lock is rendered supple and easily disentangles. On dry hair, the lock easily disentangles and the feel is smooth.

Similar cosmetic compositions can be prepared comprising the compounds of Examples 10, 11 and 12.

### Composition 3:

| | |
|---|---|
| Compound of Example 9 | 1.5% |
| Cetearyl alcohol (C₁₆/C₁₈ 50/50) | 3% |
| (Lanette O gold flakes from Cognis) | |
| Myristyl/cetyl/stearyl | 0.5% |
| myristate/palmitate/stearate mixture | |
| (Crodamol MS-V from Croda) | |
| PDMS possessing aminoethyliminopropyl | 2% |
| groups as an emulsion (DC 939 | |
| emulsion from Dow Corning) | |
| Citric acid | q.s. pH 2.5 |
| Water | q.s. for 100% |

Similar cosmetic compositions can be prepared comprising the compounds of Examples 10, 11 and 12.

### Composition 4:

| | |
|---|---|
| Compound of Example 9 | 1.5% |
| Cetearyl alcohol (C₁₆/C₁₈ 50/50) | 3% |
| (Lanette O gold flakes from Cognis) | |
| Myristyl/cetyl/stearyl | 0.5% |
| myristate/palmitate/stearate mixture | |
| (Crodamol MS-V from Croda) | |
| Avocado oil | 2% |
| Citric acid | q.s. pH 2.5 |
| Water | q.s. for 100% |

Similar cosmetic compositions can be prepared comprising the compounds of Examples 10 to 12.

### Composition 5:

| | |
|---|---|
| Compound of Example 9 | 1.5% |
| Cetearyl alcohol (C₁₆/C₁₈ 50/50) | 3% |
| (Lanette O gold flakes from Cognis) | |
| Myristyl/cetyl/stearyl | 0.5% |
| myristate/palmitate/stearate mixture | |
| (Crodamol MS-V from Croda) | |
| PDMS possessing aminoethyliminopropyl | 2% |
| groups as an emulsion (DC 939 | |
| emulsion from Dow Corning) | |
| Avocado oil | 1% |
| Citric acid | q.s. pH 2.5 |
| Water | q.s. for 100% |

Similar cosmetic compositions can be prepared comprising the compounds of Examples 10, 11 and 12.

### Composition 6:

| | |
|---|---|
| Compound of Example 9 | 3.25% |
| Cetearyl alcohol (C₁₆/C₁₈ 50/50) | 5.5% |
| (Lanette O gold flakes from Cognis) | |
| Citric acid | q.s. pH 2.5 |
| Water | q.s. for 100% |

This composition contributes a smooth feel to wet hair and the lock is rendered supple and easily disentangles. On dry hair, the lock easily disentangles and the feel is smooth.

Similar cosmetic compositions can be prepared comprising the compounds of Examples 10, 11 and 12.

### Composition 7:

| | |
|---|---|
| Compound of Example 9 | 3.25% |
| Cetearyl alcohol (C₁₆/C₁₈ 50/50) | 5.5% |
| (Lanette O gold flakes from Cognis) | |
| Myristyl/cetyl/stearyl | 1% |
| myristate/palmitate/stearate mixture | |
| (Crodamol MS-V from Croda) | |
| Citric acid | q.s. pH 2.5 |
| Water | q.s. for 100% |

This composition contributes a smooth feel to wet hair and the lock is rendered supple and easily disentangles. On dry hair, the lock easily disentangles and the feel is smooth.

Similar cosmetic compositions can be prepared comprising the compounds of Examples 10, 11 and 12.

### Composition 8:

| | |
|---|---|
| Compound of Example 9 | 3.25% |
| Cetearyl alcohol (C₁₆/C₁₈ 50/50) | 5.5% |
| (Lanette O gold flakes from Cognis) | |
| Myristyl/cetyl/stearyl | 1% |
| myristate/palmitate/stearate mixture | |
| (Crodamol MS-V from Croda) | |
| PDMS possessing aminoethyliminopropyl | 4% |
| groups as an emulsion (DC 939 | |
| emulsion from Dow Corning) | |
| Citric acid | q.s. pH 2.5 |
| Water | q.s. for 100% |

Similar cosmetic compositions can be prepared comprising the compounds of Examples 10, 11 and 12.

### Composition 9:

| | |
|---|---|
| Compound of Example 9 | 3.25% |
| Cetearyl alcohol (C₁₆/C₁₈ 50/50) | 5.5% |
| (Lanette O gold flakes from Cognis) | |
| Myristyl/cetyl/stearyl | 1% |
| myristate/palmitate/stearate mixture | |
| (Crodamol MS-V from Croda) | |
| Olive oil | 2% |
| Citric acid | q.s. pH 2.5 |
| Water | q.s. for 100% |

Similar cosmetic compositions can be prepared comprising the compounds of Examples 10, 11 and 12.

### Composition 10:

| | |
|---|---|
| Compound of Example 9 | 3.25% |
| Cetearyl alcohol (C₁₆/C₁₈ 50/50) | 5.5% |
| (Lanette O gold flakes from Cognis) | |
| Myristyl/cetyl/stearyl | 1% |
| myristate/palmitate/stearate mixture | |
| (Crodamol MS-V from Croda) | |
| PDMS possessing aminoethyliminopropyl | 4% |
| groups as an emulsion (DC 939 | |
| emulsion from Dow Corning) | |
| Olive oil | 1% |
| Citric acid | q.s. pH 2.5 |
| Water | q.s. for 100% |

Similar cosmetic compositions can be prepared comprising the compounds of Examples 10, 11 and 12.

### Composition 11:

| | |
|---|---|
| Compound of Example 9 or compound of | 5% |
| Example 12 | |
| Cetearyl alcohol (C₁₆/C₁₈ 50/50) | 8% |
| (Lanette O gold flakes from Cognis) | |
| Citric acid | q.s. pH 2.5 |
| Water | q.s. for 100% |

These compositions contribute a smooth feel to wet hair and the lock is rendered supple and easily disentangles. On dry hair, the lock disentangles easily and the feel is smooth.

Similar cosmetic compositions can be prepared comprising the compounds of Examples 10 and 11.

### Composition 12:

| | |
|---|---|
| Compound of Example 9 | 5% |
| Cetearyl alcohol (C₁₆/C₁₈ 50/50) | 8% |
| (Lanette O gold flakes from Cognis) | |
| Myristyl/cetyl/stearyl | 1.5% |
| myristate/palmitate/stearate mixture | |
| (Crodamol MS-V from Croda) | |
| Citric acid | q.s. pH 2.5 |
| Water | q.s. for 100% |

This composition contributes a smooth feel to wet hair and the lock is rendered supple and easily disentangles. On dry hair, the lock easily disentangles and the feel is smooth.

Similar cosmetic compositions can be prepared comprising the compounds of Examples 10, 11 and 12.

### Composition 13:

| | |
|---|---|
| Compound of Example 9 | 5% |
| Cetearyl alcohol (C₁₆/C₁₈ 50/50) | 8% |
| (Lanette O gold flakes from Cognis) | |
| Myristyl/cetyl/stearyl | 1.5% |
| myristate/palmitate/stearate mixture | |
| (Crodamol MS-V from Croda) | |
| PDMS possessing aminoethyliminopropyl | 4% |
| groups as an emulsion (DC 939 | |
| emulsion from Dow Corning) | |
| Citric acid | q.s. pH 2.5 |
| Water | q.s. for 100% |

Similar cosmetic compositions can be prepared comprising the compounds of Examples 10, 11 and 12.

### Composition 14:

| | |
|---|---|
| Compound of Example 9 | 5% |
| Cetearyl alcohol (C₁₆/C₁₈ 50/50) | 8% |
| (Lanette O gold flakes from Cognis) | |
| Myristyl/cetyl/stearyl | 1.5% |
| myristate/palmitate/stearate mixture | |
| (Crodamol MS-V from Croda) | |
| Apricot oil | 2% |
| Citric acid | q.s. pH 2.5 |
| Water | q.s. for 100% |

Similar cosmetic compositions can be prepared comprising the compounds of Examples 10, 11 and 12.

### Composition 15:

| | |
|---|---|
| Compound of Example 9 | 5% |
| Cetearyl alcohol (C₁₆/C₁₈ 50/50) | 8% |
| (Lanette O gold flakes from Cognis) | |
| Myristyl/cetyl/stearyl | 1.5% |
| myristate/palmitate/stearate mixture | |
| (Crodamol MS-V from Croda) | |
| PDMS possessing aminoethyliminopropyl | 4% |
| groups as an emulsion (DC 939 | |
| emulsion from Dow Corning) | |
| Apricot oil | 1% |
| Citric acid | q.s. pH 2.5 |
| Water | q.s. for 100% |

Similar cosmetic compositions can be prepared comprising the compounds of Examples 10, 11 and 12.

### Composition 16:

| | |
|---|---|
| Compound of Example 9 | 1.5% |
| Cetearyl alcohol (C₁₆/C₁₈ 50/50) | 8% |
| (Lanette O gold flakes from Cognis) | |
| Citric acid | q.s. pH 2.5 |
| Water | q.s. for 100% |

This composition contributes a smooth feel to wet hair and the lock is rendered supple and easily disentangles. On dry hair, the lock easily disentangles and the feel is smooth.

Similar cosmetic compositions can be prepared comprising the compounds of Examples 10, 11 and 12.

### Composition 17:

| | |
|---|---|
| Compound of Example 9 | 1.5% |
| Cetearyl alcohol (C₁₆/C₁₈ 50/50) | 8% |
| (Lanette O gold flakes from Cognis) | |
| Myristyl/cetyl/stearyl | 1.5% |
| myristate/palmitate/stearate mixture | |
| (Crodamol MS-V from Croda) | |
| Citric acid | q.s. pH 2.5 |
| Water | q.s. for 100% |

Similar cosmetic compositions can be prepared comprising the compounds of Examples 10, 11 and 12.

### Composition 18:

| | |
|---|---|
| Compound of Example 9 | 1.5% |
| Cetearyl alcohol (C₁₆/C₁₈ 50/50) | 8% |
| (Lanette O gold flakes from Cognis) | |
| Myristyl/cetyl/stearyl | 1.5% |
| myristate/palmitate/stearate mixture | |
| (Crodamol MS-V from Croda) | |
| PDMS possessing aminoethyliminopropyl | 4% |
| groups as an emulsion (DC 939 | |
| emulsion from Dow Corning) | |
| Citric acid | q.s. pH 2.5 |
| Water | q.s. for 100% |

Similar cosmetic compositions can be prepared comprising the compounds of Examples 10, 11 and 12.

### Composition 19:

| | |
|---|---|
| Compound of Example 9 | 1.5% |
| Cetearyl alcohol (C₁₆/C₁₈ 50/50) | 8% |
| (Lanette O gold flakes from Cognis) | |
| Myristyl/cetyl/stearyl | 1.5% |
| myristate/palmitate/stearate mixture | |
| (Crodamol MS-V from Croda) | |
| Argan oil | 3% |
| Citric acid | q.s. pH 2.5 |
| Water | q.s. for 100% |

Similar cosmetic compositions can be prepared comprising the compounds of Examples 10, 11 and 12.

### Composition 20:

| | |
|---|---|
| Compound of Example 9 | 1.5% |
| Cetearyl alcohol (C₁₆/C₁₈ 50/50) | 8% |
| (Lanette O gold flakes from Cognis) | |
| Myristyl/cetyl/stearyl | 1.5% |
| myristate/palmitate/stearate mixture | |
| (Crodamol MS-V from Croda) | |
| PDMS possessing aminoethyliminopropyl | 4% |
| groups as an emulsion (DC 939 | |
| emulsion from Dow Corning) | |
| Argan oil | 0.5% |
| Citric acid | q.s. pH 2.5 |
| Water | q.s. for 100% |

Similar cosmetic compositions can be prepared comprising the compounds of Examples 10, 11 and 12.

### Composition 21:

| | |
|---|---|
| Compound of Example 9 | 5% |
| Cetearyl alcohol (C₁₆/C₁₈ 50/50) | 5% |
| (Lanette O gold flakes from Cognis) | |
| Citric acid | q.s. pH 2.5 |
| Water | q.s. for 100% |

This composition contributes a smooth feel to wet hair and the lock is rendered supple and easily disentangles. On dry hair, the lock easily disentangles and the feel is smooth.

Similar cosmetic compositions can be prepared comprising the compounds of Examples 10, 11 and 12.

### Composition 22:

| | |
|---|---|
| Compound of Example 9 | 5% |
| Cetearyl alcohol (C₁₆/C₁₈ 50/50) | 5% |
| (Lanette O gold flakes from Cognis) | |
| Myristyl/cetyl/stearyl | 1% |
| myristate/palmitate/stearate mixture | |
| (Crodamol MS-V from Croda) | |
| Citric acid | q.s. pH 2.5 |
| Water | q.s. for 100% |

Similar cosmetic compositions can be prepared comprising the compounds of Examples 10, 11 and 12.

### Composition 23:

| | |
|---|---|
| Compound of Example 9 | 5% |
| Cetearyl alcohol (C₁₆/C₁₈ 50/50) | 5% |
| (Lanette O gold flakes from Cognis) | |
| Myristyl/cetyl/stearyl | 1% |
| myristate/palmitate/stearate mixture | |
| (Crodamol MS-V from Croda) | |
| PDMS possessing aminoethyliminopropyl | 3% |
| groups as an emulsion (DC 939 | |
| emulsion from Dow Corning) | |
| Citric acid | q.s. pH 2.5 |
| Water | q.s. for 100% |

Similar cosmetic compositions can be prepared comprising the compounds of Examples 10, 11 and 12.

### Composition 24:

| | |
|---|---|
| Compound of Example 9 | 5% |
| Cetearyl alcohol (C₁₆/C₁₈ 50/50) | 5% |
| (Lanette O gold flakes from Cognis) | |
| Myristyl/cetyl/stearyl | 1% |
| myristate/palmitate/stearate mixture | |
| (Crodamol MS-V from Croda) | |
| Jojoba oil | 2% |
| Citric acid | q.s. pH 2.5 |
| Water | q.s. for 100% |

Similar cosmetic compositions can be prepared comprising the compounds of Examples 10, 11 and 12.

### Composition 25:

| | |
|---|---|
| Compound of Example 9 | 5% |
| Cetearyl alcohol (C₁₆/C₁₈ 50/50) | 5% |
| (Lanette O gold flakes from Cognis) | |
| Myristyl/cetyl/stearyl | 1% |
| myristate/palmitate/stearate mixture | |
| (Crodamol MS-V from Croda) | |
| PDMS possessing aminoethyliminopropyl | 3% |
| groups as an emulsion (DC 939 | |
| emulsion from Dow Corning) | |
| Jojoba oil | 1.5% |
| Citric acid | q.s. pH 2.5 |
| Water | q.s. for 100% |

Similar cosmetic compositions can be prepared comprising the compounds of Examples 10, 11 and 12.

### Composition 26:

| | |
|---|---|
| Compound of Example 9 | 5% |
| Cetearyl alcohol (C₁₆/C₁₈ 50/50) | 3% |
| (Lanette O gold flakes from Cognis) | |
| Citric acid | q.s. pH 2.5 |
| Water | q.s. for 100% |

This composition contributes a smooth feel to wet hair and the lock is rendered supple and easily disentangles. On dry hair, the lock easily disentangles and the feel is smooth.

Similar cosmetic compositions can be prepared comprising the compounds of Examples 10, 11 and 12.

### Composition 27:

| | |
|---|---|
| Compound of Example 9 | 5% |
| Cetearyl alcohol (C₁₆/C₁₈ 50/50) | 3% |
| (Lanette O gold flakes from Cognis) | |
| Myristyl/cetyl/stearyl | 0.5% |
| myristate/palmitate/stearate mixture | |
| (Crodamol MS-V from Croda) | |
| Citric acid | q.s. pH 2.5 |
| Water | q.s. for 100% |

Similar cosmetic compositions can be prepared comprising the compounds of Examples 10, 11 and 12.

### Composition 28:

| | |
|---|---|
| Compound of Example 9 | 5% |
| Cetearyl alcohol (C₁₆/C₁₈ 50/50) | 3% |
| (Lanette O gold flakes from Cognis) | |
| Myristyl/cetyl/stearyl | 0.5% |
| myristate/palmitate/stearate mixture | |
| (Crodamol MS-V from Croda) | |
| PDMS possessing aminoethyliminopropyl | 1.5% |
| groups as an emulsion (DC 939 | |
| emulsion from Dow Corning) | |
| Citric acid | q.s. pH 2.5 |
| Water | q.s. for 100% |

Similar cosmetic compositions can be prepared comprising the compounds of Examples 10, 11 and 12.

### Composition 29:

| | |
|---|---|
| Compound of Example 9 | 5% |
| Cetearyl alcohol (C₁₆/C₁₈ 50/50) | 3% |
| (Lanette O gold flakes from Cognis) | |
| Myristyl/cetyl/stearyl | 0.5% |
| myristate/palmitate/stearate mixture | |
| (Crodamol MS-V from Croda) | |
| Shea butter | 0.5% |
| Citric acid | q.s. pH 2.5 |
| Water | q.s. for 100% |

Similar cosmetic compositions can be prepared comprising the compounds of Examples 10, 11 and 12.

### Composition 30:

| | |
|---|---|
| Compound of Example 9 | 5% |
| Cetearyl alcohol (C₁₆/C₁₈ 50/50) | 3% |
| (Lanette O gold flakes from Cognis) | |
| Myristyl/cetyl/stearyl | 0.5% |
| myristate/palmitate/stearate mixture | |
| (Crodamol MS-V from Croda) | |
| PDMS possessing aminoethyliminopropyl | 1.5% |
| groups as an emulsion (DC 939 | |
| emulsion from Dow Corning) | |
| Shea butter | 0.1% |
| Citric acid | q.s. pH 2.5 |
| Water | q.s. for 100% |

Similar cosmetic compositions can be prepared comprising the compounds of Examples 10, 11 and 12.

1. A method for cosmetic treatment of keratinous substances, comprising:
applying to the keratinous substance to be treated a cosmetic composition, comprising:
a physiologically acceptable medium,
at least one compound of formula (I): 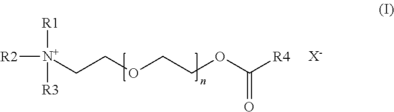
wherein
n is an integer between 1 and 10;
R1, R2 and R3 are each independently, a saturated or unsaturated, linear, cyclic or branched carbon-comprising group, comprising from 1 to 22 carbon atoms and optionally substituted by one or more hydroxyl and/or amino (—NRR′) radicals,
wherein
R and R′ are each independently H or C₁-C₆ alkyl; or
R1 is a saturated or unsaturated, linear, cyclic or branched carbon-comprising group, comprising from 1 to 22 carbon atoms and optionally substituted by one or more hydroxyl and/or amino (—NRR′) radicals, wherein R and R′ are defined above,
R2 and R3 form, with a nitrogen atom to which they are connected, a saturated or unsaturated carbon-comprising heterocycle comprising 5 or 6 ring members,
wherein one or two nonadjacent carbon atoms are optionally replaced by an oxygen, sulphur or nitrogen (—NR″) atom to form a heterocycle, wherein R″═H or C₁-C₂₂ alkyl, and
the heterocycle is optionally substituted by one or more identical or different radicals selected from the group consisting of aryl, C₁-C₂₂ alkyl, hydroxyl, and amino (—NRR′) radicals,
wherein R and R′ are independently, H or a C₁-C₆ alkyl,
R4 is selected from the group consisting of:
a linear C₁-C₃₀ or branched C₃-C₃₀ alkyl radical optionally interrupted by a 3-, 4- or 5-membered carbon-comprising ring and/or optionally substituted by (i) an aryl, itself optionally substituted by one or more identical or different radicals selected from the group consisting of C₁-C₆ alkyl, hydroxyl and amino (—NRR′) radicals, with R and R′ chosen, independently of one another, from H and C₁-C₆ alkyl, and/or by (ii) one or more identical or different radicals chosen from hydroxyl and amino (—NRR′) radicals, with R and R′ chosen, independently of one another, from H and C₁-C₆ alkyl,
a linear C₂-C₃₀ or branched C₃-C₃₀ alkenyl radical comprising one or more double bonds and optionally substituted by one or more identical or different radicals selected from the group consisting of hydroxyl and amino (—NRR′) radicals, with R and R′ chosen, independently of one another, from H and C₁-C₆ alkyl,
an aryl group optionally substituted by one or more identical or different radicals selected from the group consisting of C₁-C₂₂ alkyl, C₁-C₂₂ alkylcarbonyl, hydroxyl and amino (—NRR′) radicals, with R and R′ chosen, independently of one another, from H and C₁-C₆ alkyl;
X⁻ is an organic or inorganic anion or a mixture of organic or inorganic anions, in order to ensure the electrical neutrality of the compound of formula (I).

2. The method according to claim 1, wherein
R1=R2=ethyl;
R3 is an optionally substituted linear or branched alkyl radical comprising from 1 to 22 carbon atoms optionally substituted by one or more hydroxyl and/or amino (—NRR′) radicals, wherein R and R′ are independently H or C₁-C₆ alkyl;
R4 is selected from the group consisting of:
an optionally substituted linear C₁-C₃₀ or branched C₃-C₃₀ alkyl radical which is optionally interrupted by a 3-, 4- or 5-membered carbon-comprising ring, optionally substituted by one or more identical or different radicals chosen from hydroxyl and amino (—NRR′) radicals, wherein R and R′ are independently H or C₁-C₆ alkyl;
an optionally substituted linear or branched C₆-C₃₀ alkenyl radical comprising one or more double bonds, optionally substituted by one or more identical or different radicals chosen from hydroxyl (—OH) and amino (—NRR′) radicals, wherein R and R′ are independently H or C₁-C₆ alkyl;
an optionally substituted aryl group, optionally substituted by one or more identical or different radicals chosen from C₁-C₂₂ alkyl, C₁-C₂₂ alkylcarbonyl, hydroxyl (—OH) and amino (—NRR′) radicals, wherein R and R′ are independently H or C₁-C₆ alkyl.

3. The method according to claim 1, wherein a content of the at least one compound of formula (I) is between 0.01 and 50% by weight, with respect to the total weight of the composition.

4. The method according to claim 1, wherein the at least one compound of formula (I) is selected from the group consisting of the following compounds and their solvates:

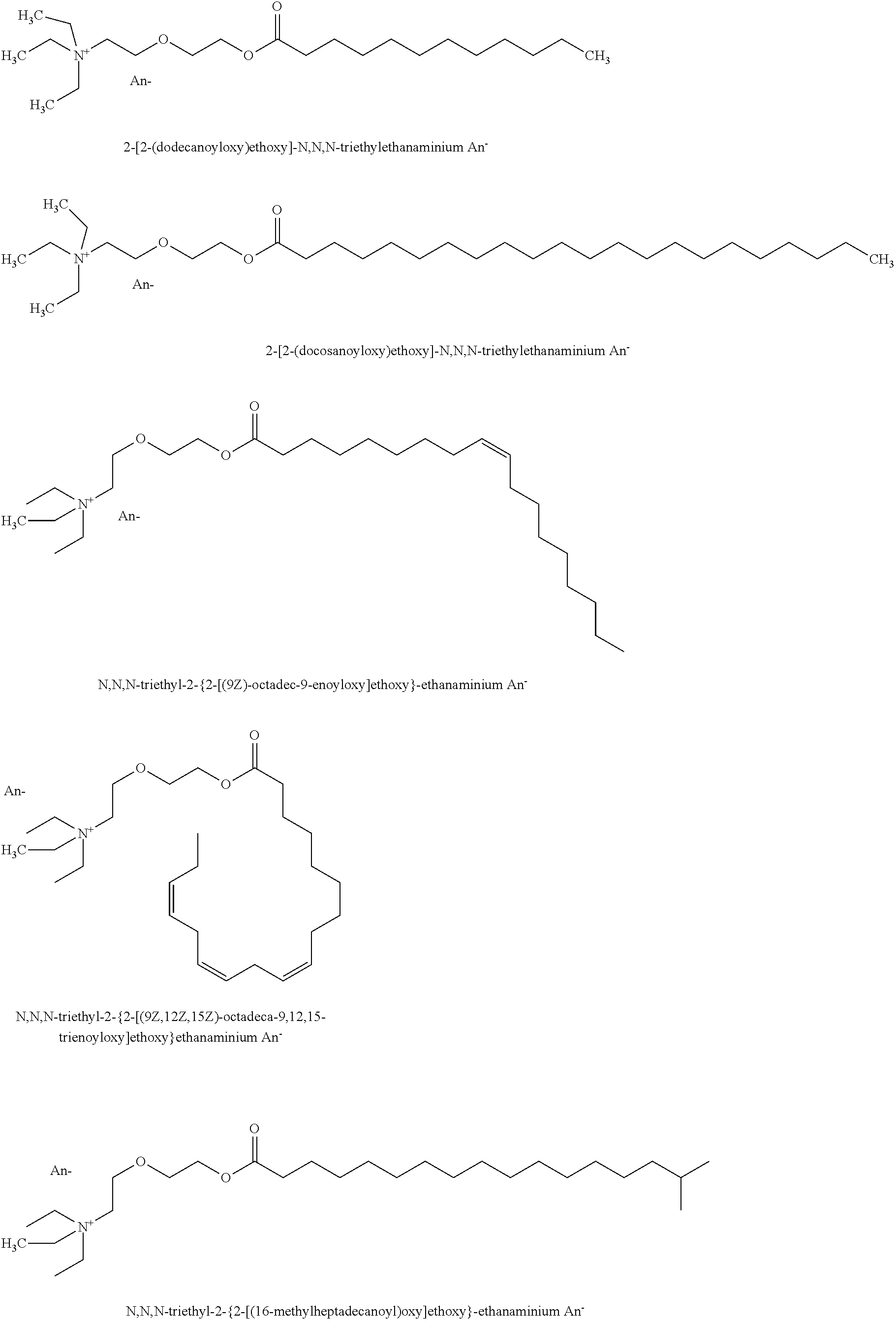

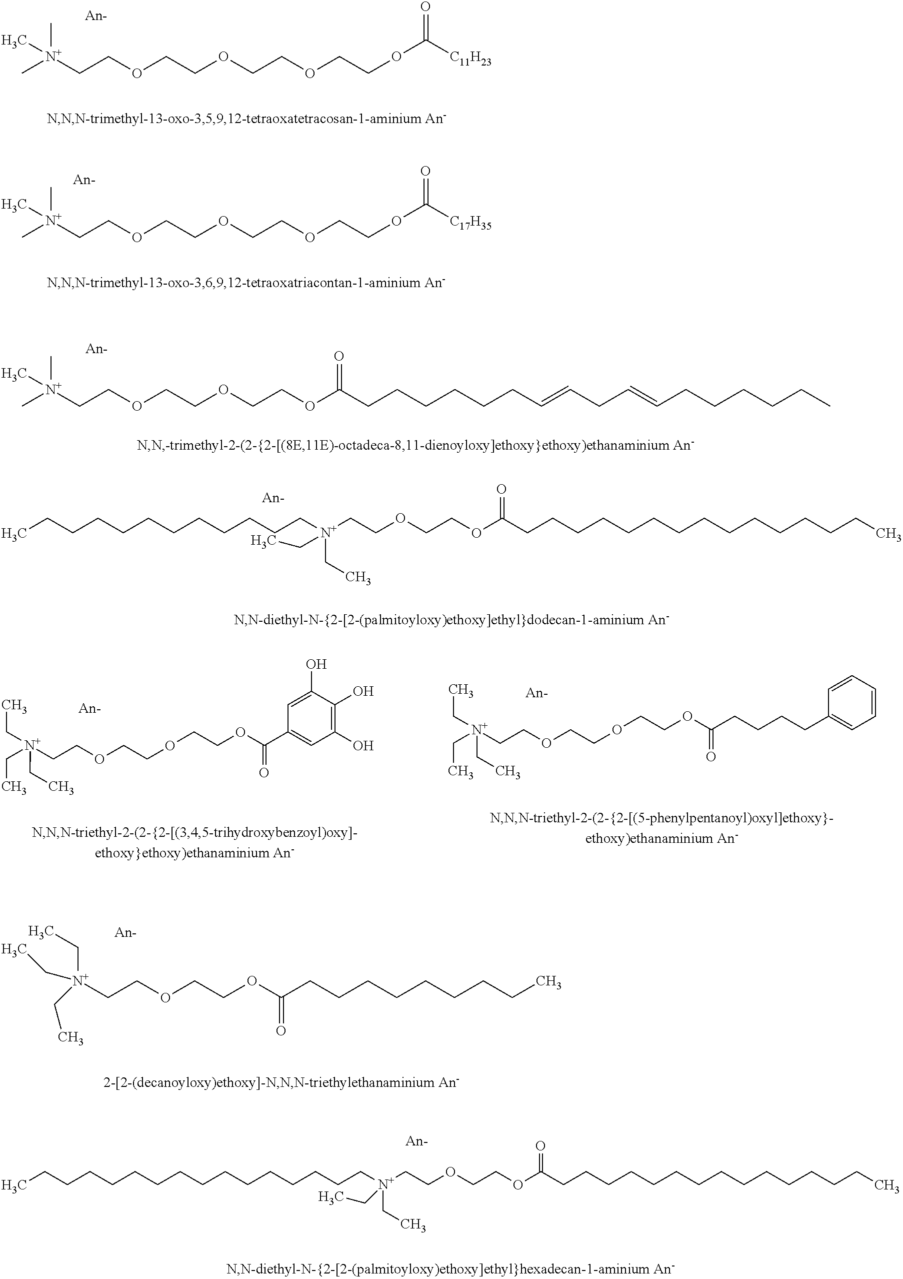

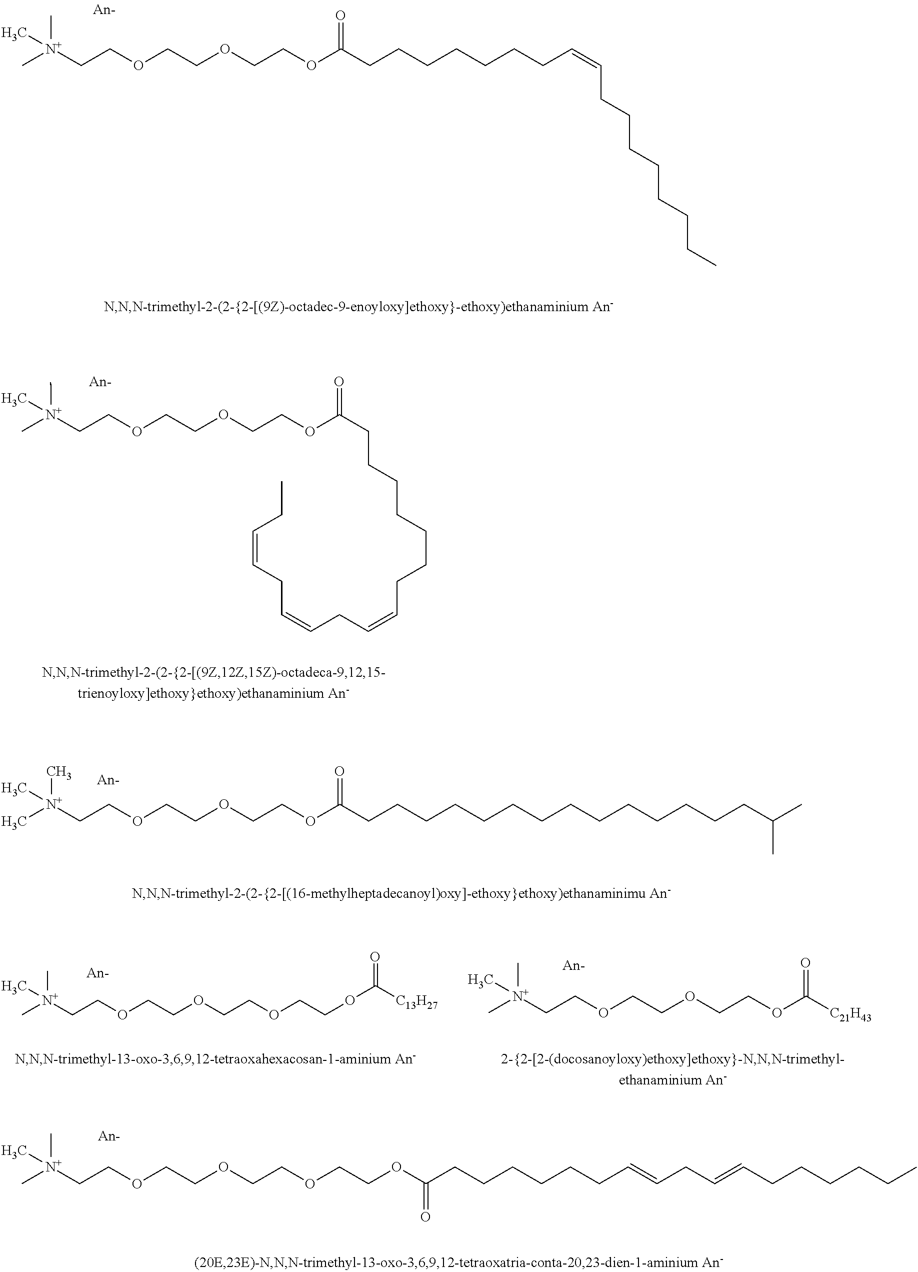

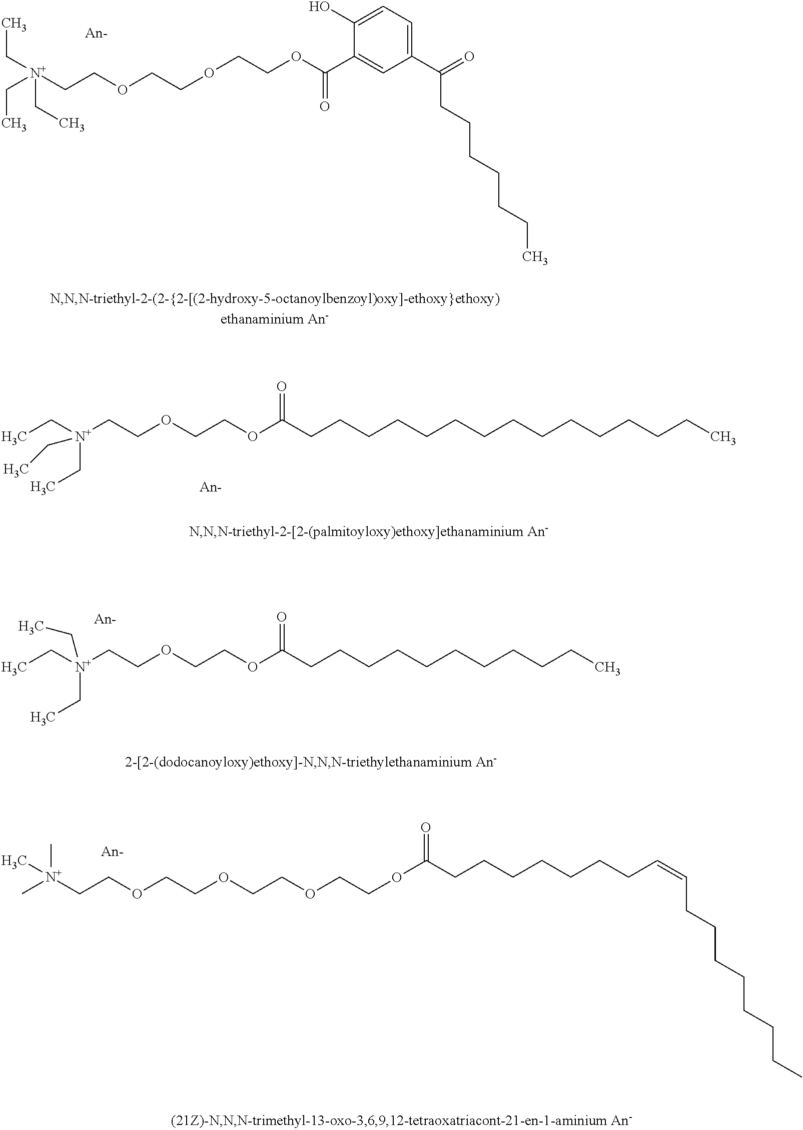

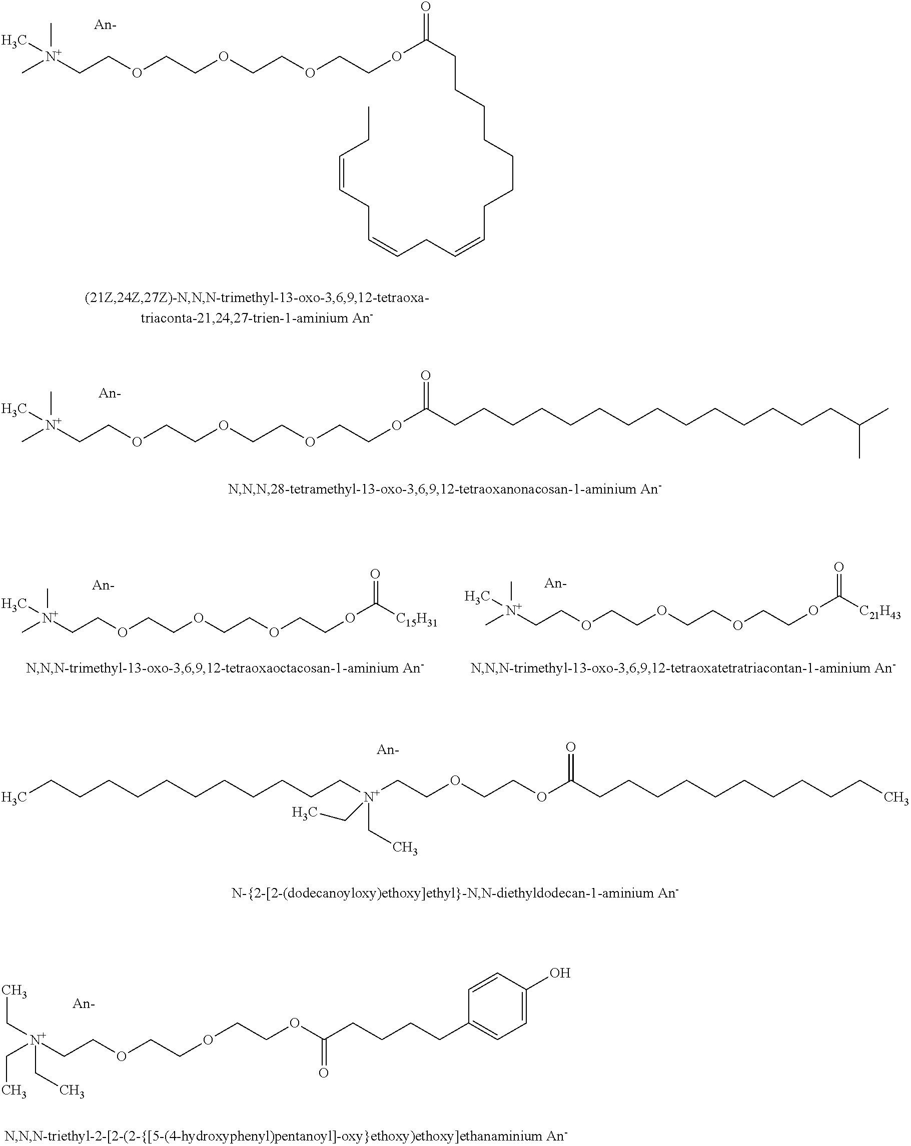

5. A method for conditioning of the hair, comprising the method according to claim 1, wherein the conditioning comprises at least one selected from the group consisting of providing the hair with suppleness, disentangling the hair, smoothing the hair, increasing the ability of the hair to be combed and improving the suppleness, the disentangling, the smoothing and the ability to be combed.

6. A hair cosmetic composition, comprising as a conditioning agent: at least one compound of formula (I) as defined in claim 1.

7. The hair cosmetic composition according to claim 6, further comprising:
a cosmetically acceptable medium;
and at least one standard cosmetic ingredient selected from the group consisting of propellants, water, carbon-comprising oils, C₈-C₄₀ esters, C₈-C₄₀ acids, C₁-C₄₀ alcohols, organic solvents, nonionic surfactants, cationic surfactants, anionic surfactants, amphoteric surfactants, zwitterionic surfactants, sunscreens, moisturizing agents, antidandruff agents, antioxidants, reducing agents, oxidation bases, couplers, oxidizing agents, direct dyes, hair-straightening agents, pearlescent and opacifying agents, plasticizing or coalescence agents, hydroxy acids, pigments, fillers, silicones, polymeric or nonpolymeric thickeners, emulsifiers and polymers.

8. A compound of formula (Ia): 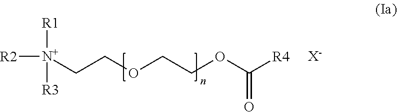
wherein
n is an integer between 1 and 10;
R1=R2=ethyl;
R3 is an optionally substituted linear or branched alkyl radical comprising from 1 to 22 carbon atoms optionally substituted by one or more hydroxyl (—OH) and/or amino (—NRR′) radicals, wherein R and R′ are independently H or C₁-C₆ alkyl;
R4 is selected from the group consisting of:
a linear C₁-C₃₀ or branched C₃-C₃₀ alkyl radical optionally interrupted by a 3-, 4- or 5-membered carbon-comprising ring and/or optionally substituted by (i) an aryl, itself optionally substituted by one or more identical or different radicals selected from the group consisting of C₁-C₆ alkyl, hydroxyl and amino (—NRR′) radicals, with R and R′ chosen, independently of one another, from H and C₁-C₆ alkyl, and/or by (ii) one or more identical or different radicals chosen from hydroxyl and amino (—NRR′) radicals, with R and R′ chosen, independently of one another, from H and C₁-C₆ alkyl,
a linear C₂-C₃₀ or branched C₃-C₃₀ alkenyl radical comprising one or more double bonds and optionally substituted by one or more identical or different radicals selected from the group consisting of hydroxyl and amino (—NRR′) radicals, with R and R′ chosen, independently of one another, from H and C₁-C₆ alkyl,
an aryl group optionally substituted by one or more identical or different radicals selected from the group consisting of C₁-C₂₂ alkyl, C₁-C₂₂ alkylcarbonyl, hydroxyl and amino (—NRR′) radicals, with R and R′ chosen, independently of one another, from H and C₁-C₆ alkyl;
X⁻ is an organic or inorganic anion or a mixture of organic or inorganic anions, in order to ensure the electrical neutrality of the compound of formula (I).

9. A cosmetic composition comprising, in a physiologically acceptable medium, at least one compound of formula (Ia) according to claim 8.

10. The cosmetic composition according to claim 9, wherein the physiologically acceptable medium comprises at least one standard cosmetic ingredient selected from the group consisting of propellants, water, carbon-comprising oils, C₈-C₄₀ esters, C₈-C₄₀ acids, C₁-C₄₀ alcohols, organic solvents, nonionic surfactants, cationic surfactants, anionic surfactants, amphoteric surfactants, zwitterionic surfactants, sunscreens, moisturizing agents, antidandruff agents, antioxidants, reducing agents, oxidation bases, couplers, oxidizing agents, direct dyes, hair-straightening agents, pearlescent and opacifying agents, plasticizing or coalescence agents, hydroxy acids, pigments, fillers, silicones, polymeric or nonpolymeric thickeners, emulsifiers and polymers.

11. The cosmetic composition according to claim 7 wherein a content of the at least one compound of formula (I) is between 0.01 and 50% by weight, with respect to the total weight of the composition.

12. The cosmetic composition according to claim 7 wherein the at least one compound of formula (I) is selected from the group consisting of the following compounds and their solvates:

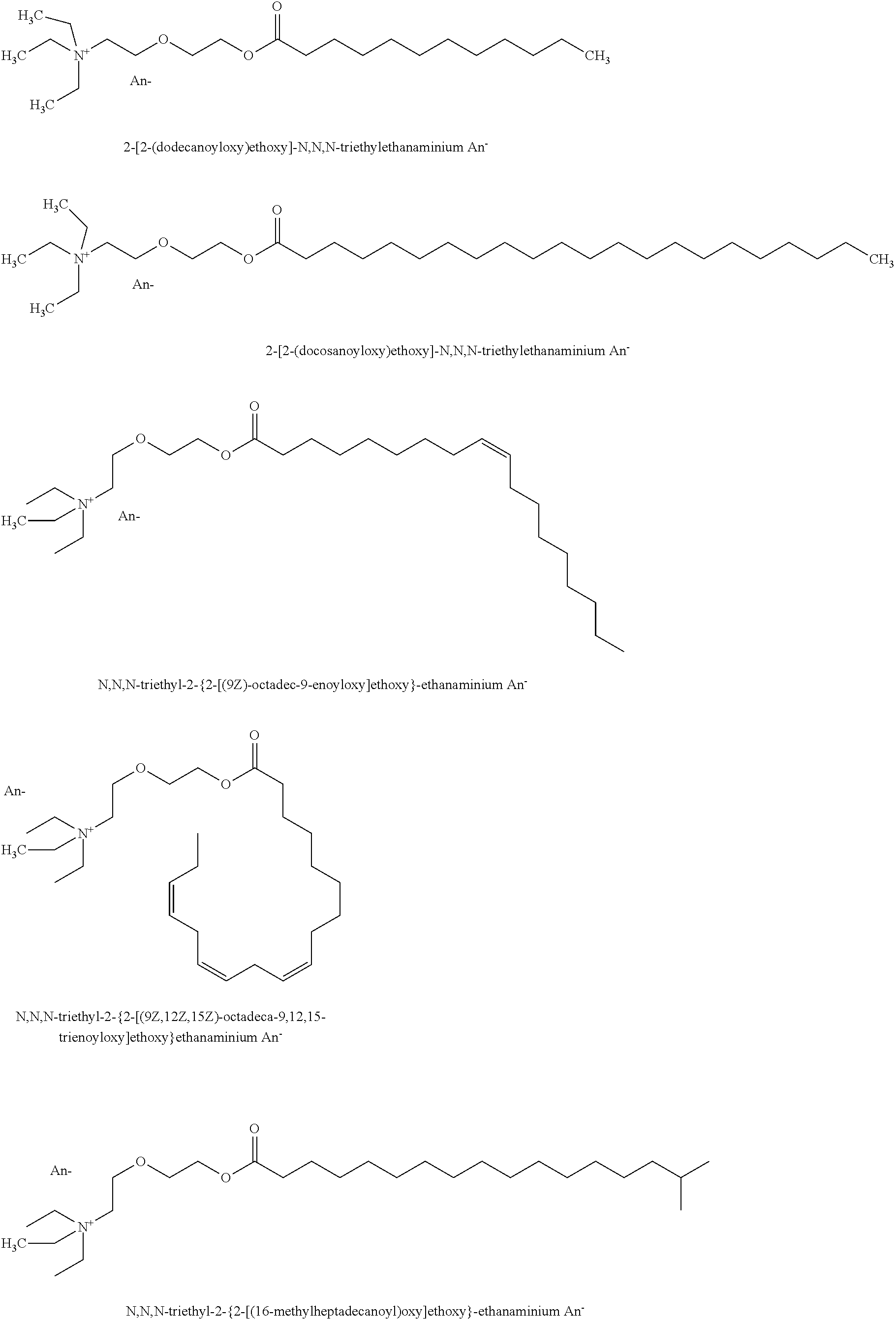

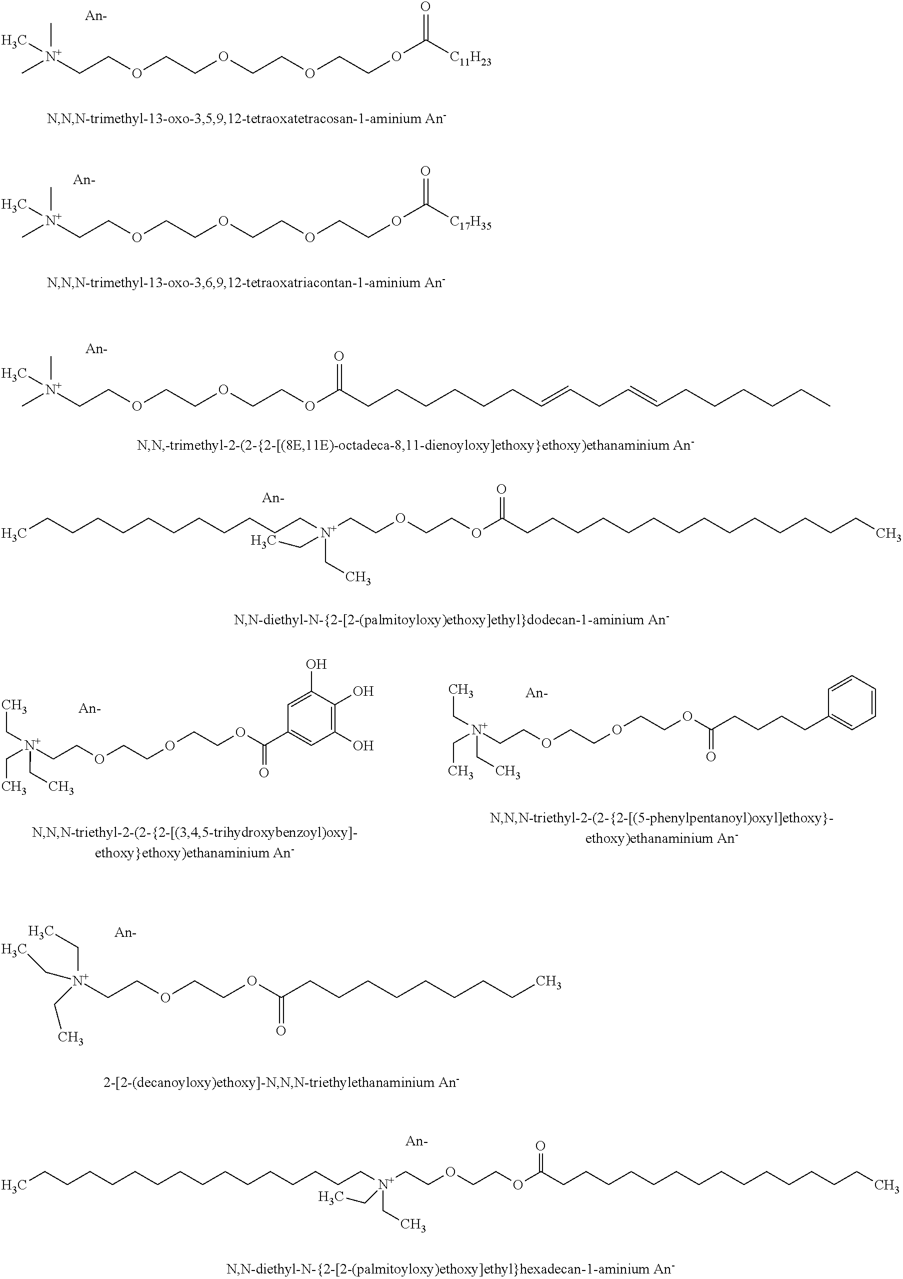

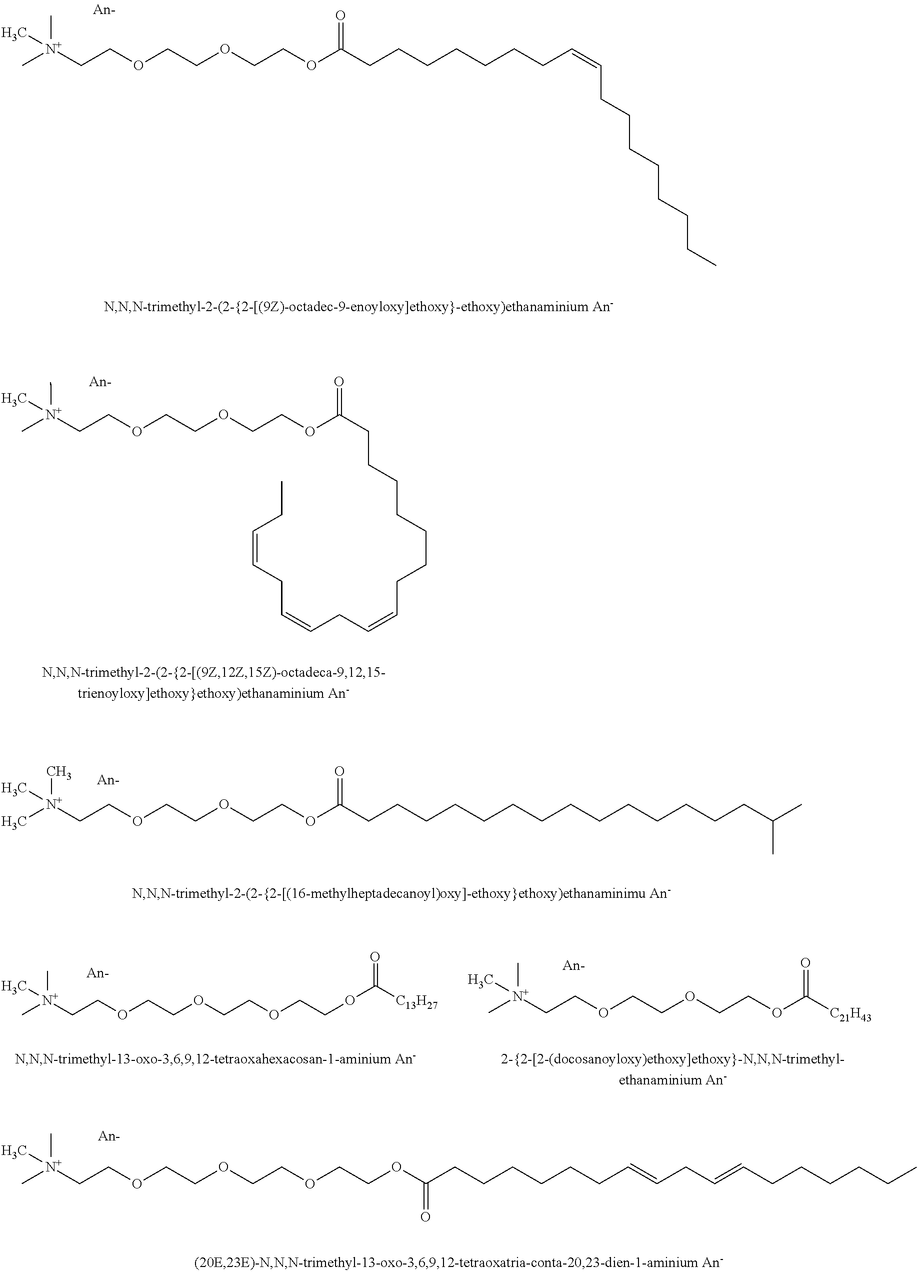

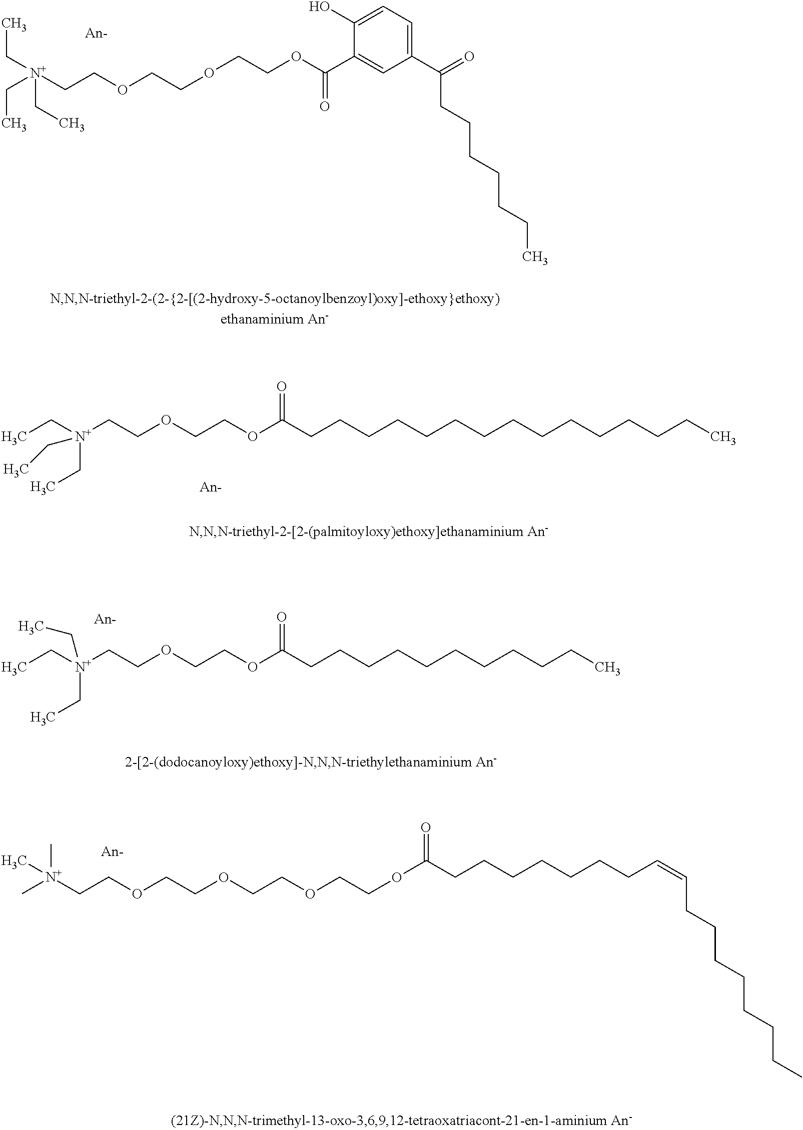

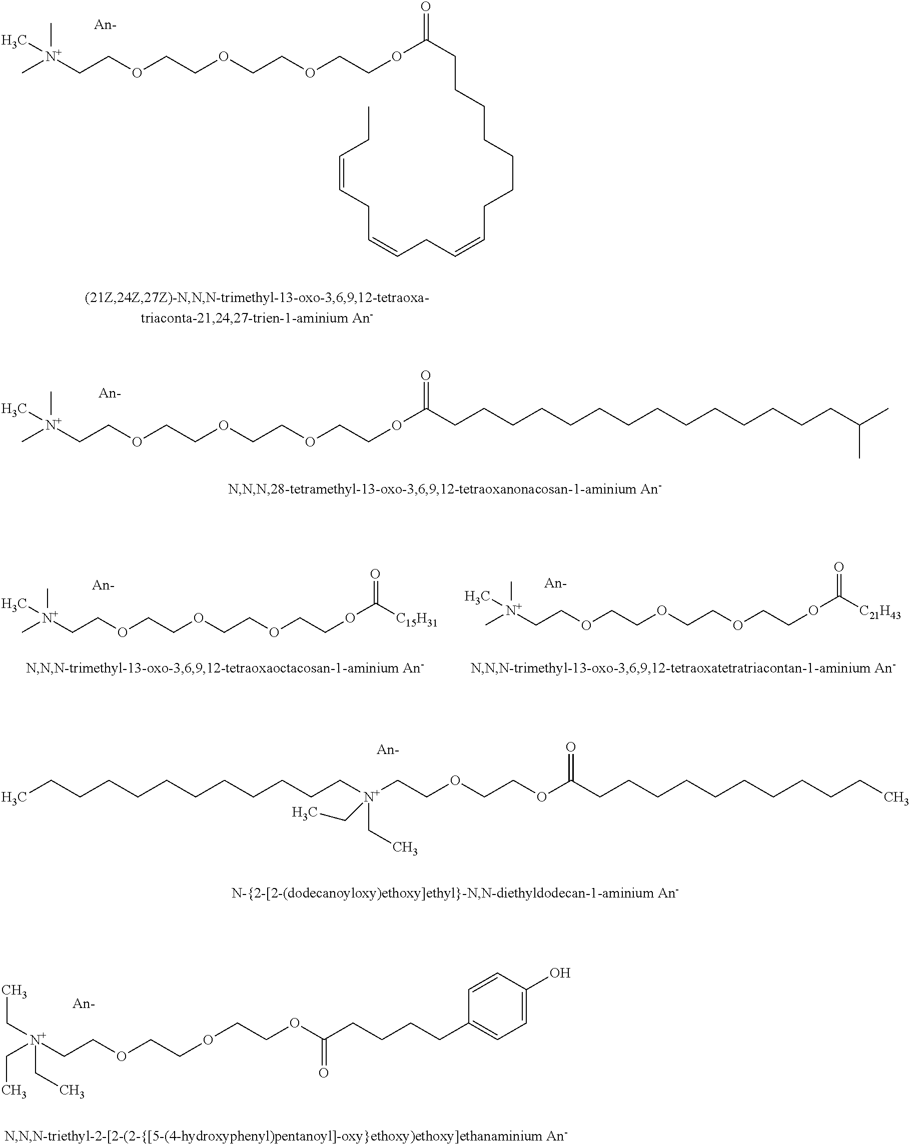

13. The cosmetic composition according to claim 7, which comprises at least one C₈-C₄₀ fatty alcohol, wherein a content of the at least one fatty alcohol is 1 to 15% by weight of the cosmetic composition.

14. A product comprising the cosmetic composition according to one of claims claim 7, wherein the product is for at least one purpose selected from the group consisting of caring for, cleaning and/or making up the skin of the body or of the face, the lips, the eyebrows, the eyelashes, the nails and the hair, an antisun or self-tanning product, a body hygiene product and a hair product for caring for, cleaning, styling, shaping or dyeing the hair.

15. The product according to claim 14, which is for the care and the cosmetic treatment of weakened and/or damaged hair.
